(19) United States
(12) Patent Application Publication
(10) Pub. No.: US 2025/0339475 A1
(43) Pub. Date: Nov. 6, 2025

(54) HEPATOPROTECTIVE PROBIOTIC COMPOSITIONS

(71) Applicant: Board of Regents, The University of Texas System, Austin, TX (US)

(72) Inventors: Laura Beretta, Austin, TX (US); Suet Ying Kwan, Austin, TX (US)

(21) Appl. No.: 19/128,118

(22) PCT Filed: Nov. 9, 2023
(86) PCT No.: PCT/US2023/037111

§ 371 Date: May 7, 2025

Related U.S. Application Data
(60) Provisional application No. 63/424,378, filed on Nov. 10, 2022.

(51) Int. Cl. A61K 35/741; A61K 35/00; A61K 45/06; A61P 1/16; C12N 1/20; C12R 1/01

(52) U.S. Cl. CPC A61K 35/741; A61K 45/06; A61P 1/16; C12N 1/205; A61K 2035/115; C12R 2001/01

(57) ABSTRACT

The present disclosure provides compositions with probiotic microbes, including one or more of Bacteroides finegoldii, Bacteroides caccae, Bacteroides ovatus, Bacteroides uniformis, Parabacteroides distasonis, Alistipes onderdonkii, Anaerobutyricum hallii, or Gemmiger formicilis, that provide hepatoprotective activity in a subject. Further disclosed herein are methods for determining whether a subject has or is at risk of developing liver disease and associated methods for monitoring liver disease progression during treatment.

### CROSS REFERENCE TO RELATED APPLICATIONS

The present application claims the benefit of priority under U.S.C. § 119(e) to U.S. Provisional Application Ser. No. 63/424,378, filed on Nov. 10, 2022, the entire contents of which is incorporated herein by reference in its entirety.

### STATEMENT OF GOVERNMENT RIGHTS

This invention was made with government support under CA217674 awarded by the National Institutes of Health. The government has certain rights in the invention.

### BACKGROUND

Nonalcoholic fatty liver disease (NAFLD) affects 25% of the adult population worldwide and encompasses a spectrum of diseases, ranging from simple steatosis to non-alcoholic steatohepatitis (NASH). Patients with NASH can further develop liver fibrosis and eventually cirrhosis and hepatocellular carcinoma (HCC). While NAFLD is associated with an extensive set of comorbidities, the presence of liver fibrosis is the most important determinant of mortality in NAFLD patients.

Animal studies have suggested a causative role for the gut microbiome in NAFLD development and progression. The absence of gut microbiota in germ-free mice conferred resistance against high-fat diet-induced hepatic steatosis. Similarly, antibiotic suppression of gut microbial growth attenuated liver inflammation and liver fibrosis in mice fed a high-fat/cholesterol/fructose diet. Several studies have also demonstrated that susceptibility to NAFLD is transmissible between mice through gut microbiota transplantation or cohousing.

On the basis of these animal studies, multiple mechanisms have been proposed for how the gut microbiome may contribute to liver disease. As the liver is the first organ exposed to gut-derived microbes and microbial-derived products and metabolites via the portal vein, it is postulated that the microbiome can induce liver inflammation. Often implicated within these models, lipopolysaccharide (LPS), a cell component of gram-negative bacteria and a known agonist of pro-inflammatory toll-like receptor 4 (TLR4), is thought to induce hepatic inflammation, a hallmark of NASH. As the human digestive tract is abundant with LPS-rich, Gram-negative bacteria, such models could suggest an inimical relationship between gut microbial health and NAFLD management and underscores the need for means of altering gut microbiomes to promote liver health.

### SUMMARY

The present disclosure provides hepatoprotective and NAFLD-inhibitory bacterial compositions. Leveraging the seminal discovery that Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis inhibit liver fibrosis in populations with high-prevalences of NAFLD and NASH, the present disclosure provides compositions of these bacteria, as well as associated methods of administration, hepatoprotection, disease prevention, and disease treatment. These bacteria exhibit efficacy when administered as single strain or as consortia and can promote long-term positive changes in gut microbial composition and health.

In a first embodiment, the present disclosure provides a composition including two or more bacteria selected from the group consisting of: Bacteroides finegoldii, Bacteroides caccae, Bacteroides ovatus, Bacteroides umformis, Parabacteroides distasonis, Alistipes onderdonkii, Anaerobutyricum hallii, and a combination thereof. In one aspect, the composition comprises at least three of the bacteria. In one aspect, the composition comprises at least four of the bacteria. In one aspect, the composition comprises at least five of the bacteria. In one aspect, the composition comprises at least six of the bacteria. In one aspect, the composition comprises at least seven of the bacteria. In one aspect, the composition comprises at least eight of the bacteria.

In one aspect, the composition comprises a functional mutant of the Bacteroides finegoldii, a functional mutant of the Bacteroides caccae, a functional mutant of the Bacteroides ovatus, a functional mutant of the Bacteroides umformis, a functional mutant of the Parabacteroides distasonis, a functional mutant of the Alistipes onderdonkii, a functional mutant of the Anaerobutyricum hallii, a functional mutant of the Gemmiger formicilis, or a combination thereof.

In one aspect, the Bacteroides finegoldii comprises at least 90% identity to the nucleotide sequence of strain DSM 17565 deposited with the Deutsche Sammlung von Mikroorganismen und Zellkulturen (DSMZ); the Bacteroides caccae comprises at least 90% identity to the nucleotide sequence of strain DSM 19024 deposited with the DSMZ; the Bacteroides ovatus comprises at least 90% identity to the nucleotide sequence of strain 1896 DSM deposited with the DSMZ; the Bacteroides uniformis comprises at least 90% identity to the nucleotide sequence of strain DSM 6597 deposited with the DSMZ; the Parabacteroides distasonis comprises at least 90% identity to the nucleotide sequence of strain DSM 20701 deposited with the DSMZ; the Alistipes onderdonkii comprises at least 90% identity to the nucleotide sequence of strain DSM 32839 deposited with the DSMZ; the Anaerobutyricum hallii comprises at least 90% identity to the nucleotide sequence of strain DSM 3353 deposited with the DSMZ; the Gemmiger formicilis comprises at least 90% identity to the nucleotide sequence of strain ATCC 27749 deposited with the American Type Culture Collection (ATCC); or a combination thereof. In one aspect, the identity is at least 91% identity, at least 92% identity, at least 93% identity, at least 94% identity, at least 95% identity, at least 96% identity, at least 97% identity, at least 98% identity, or at least 99% identity. In one aspect, the composition comprises between about 1×10⁷ and 1×10¹⁰ colony forming units (CFU) of live bacteria. In one aspect, a relative abundance of Bacteroides finegoldii among bacteria of the composition is between about 8% and 30%; a relative abundance of Bacteroides caccae among the bacteria of the composition is between about 11% and 45%; a relative abundance of Bacteroides ovatus among the bacteria of the composition is between about 7% and 27%; a relative abundance of Bacteroides uniformis among the bacteria of the composition is between about 3% and 10%; a relative abundance of Parabacteroides distasonis among the bacteria of the composition is between about 8% and 31%; a relative abundance of Alistipes onderdonkii among the bacteria of the composition is between about 5% and 21%; a relative abundance of Anaerobutyricum hallii among the bacteria of the composition is between about 1% and 6%; a relative abundance of Gemmiger formicilis among the bacteria of the composition is between about 4% and 16%; or a combination thereof. In one aspect, at least about 50% of bacteria of the composition are Gram-negative.

In one aspect, the composition is formulated for oral administration. In one aspect, the composition is formulated as an oral gavage, as a solid food, or as a beverage.

In another embodiment, the present disclosure provides a method of treating a disease or condition selected from steatosis, nonalcoholic steatohepatitis (NASH), nonalcoholic fatty liver disease (NAFLD), liver fibrosis, cirrhosis of the liver, or a combination thereof in a subject in need thereof including administering a composition of the present disclosure to the subject, thereby treating the disease or condition.

In one aspect, prior to the administration, the method further comprises determining the relative abundance of bacteria in the intestinal microbiome of the subject. In one aspect, the bacteria and determining is selected from: (i) determining that a relative abundance of Bacteroides finegoldii is less than about 0.2% in the intestinal microbiome in the subject; (ii) determining that a relative abundance of Bacteroides caccae is less than about 0.1% in the intestinal microbiome in the subject; (iii) determining that a relative abundance of Bacteroides ovatus is less than about 0.5% in the intestinal microbiome in the subject; (iv) determining that a relative abundance of Bacteroides uniformis is less than about 2.0% in the intestinal microbiome in the subject; (v) determining that a relative abundance of Parabacteroides distasonis is less than about 0.4% in the intestinal microbiome in the subject; (vi) determining that a relative abundance of Alistipes onderdonkii is less than about 0.2% in the intestinal microbiome in the subject; (vii) determining that a relative abundance of Anaerobutyricum hallii is less than about 1.0% in the intestinal microbiome in the subject; (viii) determining that a relative abundance of Gemmiger formicilis is less than about 0.8% in the intestinal microbiome in the subject; or (ix) a combination thereof. In one aspect, the method comprises at least two, at least three, at least four, at least five, at least six, at least seven, or each of (i)-(viii). In one aspect, the method comprises selecting the composition based on the relative abundance of the bacteria in the intestinal microbiome of the subject.

In one aspect, the administering is repeated once every 3 to 10 days. In one aspect, the composition is administered orally. In a further aspect, the method comprises administering a dietary supplement that supports the growth or maintenance of Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, or a combination thereof.

In another aspect, the method comprises administering an antimicrobial agent which kills or slows the growth of a bacterium in the subject and which does not kill or slow the growth of bacteria of the composition.

In another aspect, following the administration, the method further includes determining the relative abundance of bacteria in the intestinal microbiome of the subject, thereby determining that the disease or condition has been treated in the subject. In a particular aspect, the bacteria and determining is selected from determining that a relative abundance of Bacteroides finegoldii is greater than about 0.2% in the intestinal microbiome in the subject; determining that a relative abundance of Bacteroides caccae is greater than about 0.1% in the intestinal microbiome in the subject; determining that a relative abundance of Bacteroides ovatus is greater than about 0.5% in the intestinal microbiome in the subject; determining that a relative abundance of Bacteroides uniformis is greater than about 2.0% in the intestinal microbiome in the subject; determining that a relative abundance of Parabacteroides distasonis is greater than about 0.4% in the intestinal microbiome in the subject; determining that a relative abundance of Alistipes onderdonkii is greater than about 0.2% in the intestinal microbiome in the subject; determining that a relative abundance of Anaerobutyricum hallii is greater than about 1.0% in the intestinal microbiome in the subject; determining that a relative abundance of Gemmiger formicilis is greater than about 0.8% in the intestinal microbiome in the subject; or a combination thereof.

In a further aspect, following the administration, the methods further includes measuring less than about 0.05% relative abundance of serine among amino acids in stool of the subject, measuring greater than about 0.005% relative abundance of cysteine among amino acids in stool of the subject, measuring a ratio of cysteine to serine of greater than about 0.2 in stool of the subject, measuring a greater than 0.005% relative abundance among amino acids of homocysteine in liver tissue of the subject, measuring greater than 0.005% relative abundance among amino acids of homocysteine in stool of the subject, measuring greater than 0.05% relative abundance among amino acids of S-adenosylhomocysteine in stool of the subject, measuring greater than 4% relative abundance among amino acids of taurine in stool of the subject, measuring greater than 0.5% relative abundance among amino acids of S-adenosylhomocysteine in liver tissue of the subject, measuring less than 0.1% relative abundance among amino acids of asparagine in stool of the subject, measuring greater than 3% relative abundance among amino acids of tryptophan in stool of the subject, measuring less than 0.15% relative abundance among amino acids of kynurenine in liver tissue of the subject, measuring a kynurenine to tryptophan ratio of less than 0.05 in liver tissue of the subject, or a combination thereof, thereby determining that the disease or condition has been treated in the subject.

In an additional aspect, following the administration, the method further includes measuring greater than 10000 copies per million of enzymes from the pentose phosphate pathway non-oxidative branch in the stool of the subject, measuring greater than 12000 copies per million of enzymes from the diacylglycerol biosynthesis I pathway in the stool of the subject, measuring greater than 5000 copies per million of enzymes from the preQ0 biosynthesis pathway in the stool of the subject, measuring greater than 12000 copies per million of enzymes from the CDP-diacylglycerol biosynthesis II pathway in the stool of the subject, or a combination thereof, thereby determining that the disease or condition has been treated in the subject.

In another aspect, the method further includes measuring greater than 90 copies per million of 2-oxoglutarate in the stool of the subject, measuring greater than 1250 copies per million of cysteine synthase in the stool of the subject, measuring greater than 140 copies per million of penicillin amidase in the stool of the subject, measuring greater than 220 copies per million of N-acetylmuramic acid 6-phosphate etherase in the stool of the subject, measuring greater than 1100 copies per million of 2-C-methyl-D-erythritol 2,4-cyclodiphosphate synthase in the stool of the subject, measuring greater than 400 copies per million of L-rhamnose isomerase in the stool of the subject, or a combination thereof, thereby determining that the disease or condition has been treated in the subject.

In another embodiment, the present disclosure provides a method of identifying a subject as having a condition or being at risk of developing a condition selected from steatosis, nonalcoholic steatohepatitis (NASH), nonalcoholic fatty liver disease (NAFLD), liver fibrosis, or cirrhosis of the liver that includes measuring depletion of Bacteroides finegoldii; Bacteroides caccae; Bacteroides ovatus, Bacteroides uniformis, Parabacteroides distasonis, Alistipes onderdonkii, Anaerobutyricum hallii, Gemmiger formicilis, or a combination thereof in stool of the subject; measuring depletion of a biosynthetic pathway selected from pentose phosphate pathway non-oxidative branch, diacylglycerol biosynthesis I, preQ0 biosynthesis, or CDP-diacylglycerol biosynthesis II in stool of the subject; measuring depletion of 2-oxoglutarate synthase, cysteine synthase, penicillin amidase, N-acetylmuramic acid 6-phosphate etherase, 2-C-methyl-D-erythritol 2,-4-cyclodiphosphate synthase, L-rhamnose isomerase, or a combination thereof in stool of the subject; measuring depletion of cysteine, homocysteine, S-adenosylhomocysteine, tryptophan, or a combination thereof in stool of the subject, measuring depletion of homocysteine, S-adenosylhomocysteine, kynurenine, or a combination thereof in liver tissue of the subject, measuring an increase in serine, asparagine, or a combination thereof in stool of the subject, or a combination thereof, thereby identifying the subject as having the condition or being at risk of developing the condition.

In some aspects, the method includes measuring depletion of at least two, at least three, at least four, at least five, at least six, at least seven, or eight of Bacteroides finegoldii; Bacteroides caccae; Bacteroides ovatus, Bacteroides umformis, Parabacteroides distasonis, Alistipes onderdonkii, Anaerobutyricum hallii, Gemmiger formicilis in the stool of the subject.

In a further aspect, the method includes determining that a relative abundance of Bacteroides finegoldii is less than about 0.2% in the stool of the subject; determining that a relative abundance of Bacteroides caccae is less than about 0.1% in the stool of the subject; determining that a relative abundance of Bacteroides ovatus is less than about 0.5% in the stool of the subject; determining that a relative abundance of Bacteroides umformis is less than about 2.0% in the stool of the subject; determining that a relative abundance of Parabacteroides distasonis is less than about 0.4% in the stool of the subject; determining that a relative abundance of Alistipes onderdonkii is less than about 0.2% in the stool of the subject; determining that a relative abundance of Anaerobutyricum hallii is less than about 1.0% in the stool of the subject; determining that a relative abundance of Gemmiger formicilis is less than about 0.8% in the stool of the subject; or a combination thereof.

In another aspect, the method includes measuring less than 90 copies per million of 2-oxoglutarate in the stool of the subject, measuring less than 1250 copies per million of cysteine synthase in the stool of the subject, measuring less than 140 copies per million of penicillin amidase in the stool of the subject, measuring less than 220 copies per million of N-acetylmuramic acid 6-phosphate etherase in the stool of the subject, measuring less than 1100 copies per million of 2-C-methyl-D-erythritol 2,4-cyclodiphosphate synthase in the stool of the subject, measuring less than 400 copies per million of L-rhamnose isomerase in the stool of the subject, or a combination thereof.

In a further aspect, the method includes measuring greater than about 0.05% relative abundance of serine among amino acids in stool of the subject, measuring less than about 0.005% relative abundance of cysteine among amino acids in stool of the subject, measuring a ratio of cysteine to serine of less than about 0.2 in stool of the subject, measuring a less than 0.005% relative abundance among amino acids of homocysteine in liver tissue of the subject, measuring less than 0.005% relative abundance among amino acids of homocysteine in stool of the subject, measuring less than 0.05% relative abundance among amino acids of S-adenosylhomocysteine in stool of the subject, measuring less than 4% relative abundance among amino acids of taurine in stool of the subject, measuring less than 0.5% relative abundance among amino acids of S-adenosylhomocysteine in liver tissue of the subject, measuring greater than 0.1% relative abundance among amino acids of asparagine in stool of the subject, measuring less than 3% relative abundance among amino acids of tryptophan in stool of the subject, measuring greater than 0.15% relative abundance among amino acids of kynurenine in liver tissue of the subject, measuring a kynurenine to tryptophan ratio of at least 0.05 in liver tissue of the subject, or a combination thereof.

In an additional aspect, the method includes administering a composition of the present disclosure to the subject, thereby treating or preventing the disease or condition in the subject.

### DESCRIPTION OF THE FIGURES

The unique features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1A is a cladogram showing gut microbial species significantly depleted in subjects with liver fibrosis.

FIG. 1B is a cladogram showing gut microbial species significantly depleted in subjects with advanced liver fibrosis.

FIG. 1C is a plot of mean relative abundances of significant gut microbial taxa in subjects with and without liver fibrosis.

FIG. 1D is a plot of mean relative abundances of significant gut microbial taxa in subjects with and without advanced liver fibrosis.

FIG. 1E provides a Forest plot which summarizes risk for liver fibrosis or advanced liver fibrosis as a function of the absence or depletion of multiple gut microbial species.

FIG. 1F provides a Forest plot summarizing the protective effects of multiple gut microbial species against liver fibrosis or advanced liver fibrosis.

FIG. 2A is a box plot that summarizes histological measurements performed on germ-free mice and specific pathogen-free mice raised on normal and NASH-inducing diets.

FIG. 2B is a box plot that summarizes necropsy measurements performed on germ-free mice and specific pathogen-free mice raised on normal and NASH-inducing diets.

FIGS. 3A-3E show a series of plots of bacterial populations in samples collected from mice and a schematic of bacterial inoculation of mice. FIG. 3A is a heat map that summarizes the abundances of the 8 bacterial strains in inocula prepared and administered on days 0, 5, and 10. FIG. 3B is a series of heat maps that provides relative abundances of multiple bacterial strains in germ-free mouse stool on day 0 (immediately prior to first inoculation with a bacterial composition), and days 14 and 28 (after all 3 inoculations with bacterial compositions on days 0, 5, 10). FIG. 3C is a schematic of a mouse model study aimed to determine the effect of bacterial inoculation on liver fibrosis prevention in vivo. FIG. 3D is a plot of relative abundance of the eight bacterial strains in the liver of GF-MCD-B mice at time of necropsy. FIG. 3E is a plot showing Spearman's correlations between relative abundances of the bacterial species in the liver and in stool collected at day 28.

FIGS. 4A-4B show sets of box plots that summarize physical characteristics and health statuses of bacteria- and vehicle-treated mice. FIG. 4A is a series of box plots which summarize fibrosis stage, steatosis, inflammation, ballooning, ceroid laden macrophage counts, and NAFLD activity scores in bacteria- and vehicle-treated germ-free-MCD mice. FIG. 4B is a series of box plots which summarize liver-to-bodyweight ratios (%), colon lengths (cm), cecum weights (g), and cecum to body weight ratios (%) in bacteria- and vehicle-treated germ-free-MCD mice.

FIGS. 5A-5D show plots of metabolic pathways and enzymes associated with liver fibrosis. FIG. 5A is a heatmap of MetaCyc pathways and enzymes negatively associated with both fibrosis and advanced fibrosis in 340 cohort subjects, and enriched in the gut microbiome of GF-MCD-B after bacterial inoculation. Abundance (CPM) of these pathways and enzymes are shown for the inocula used to treat mice on days 0 (I1), 5 (I2) and 10 (I3), and median abundances in the stool of GF-MCD-B mice before (d0) and after inoculations (d14, d28). FIG. 5B is a plot of abundances of the MetaCyc pathways and enzymes significantly depleted in the gut metagenome of the cohort subjects with liver fibrosis. FIG. 5C is a Forest plot that shows the increased risk of liver fibrosis in cohort subjects when the stool abundance of the identified MetaCyc pathways and enzymes were at low abundance. FIG. 5D is a Forest plot that shows the protective effect against liver fibrosis in cohort subjects when the stool abundance of MetaCyc pathways and enzymes were present at high abundance.

FIGS. 6A-6E show plots of cysteine-related amino acids in mice subjected to bacterial inoculation or vehicle treatment or from a human cohort. FIG. 6A is a plot of stool levels of cysteine-related amino acids from the mice. FIG. 6B is a plot of hepatic levels of homocysteine and S-adenosylhomocysteine (SAH) from the mice. FIG. 6C is a plot of Spearman's correlation coefficients and p-values between liver fibrosis score and stool levels of cysteine-related amino acids in the mice. FIG. 6D is a plot of Spearman's correlation between stool abundance of Bacteroides uniformis and cysteine-to-serine ratios in stool from the mouse study. FIG. 6E is a plot of Spearman's correlation between abundance of Bacteroides uniformis and the cysteine synthase gene in stool from the human cohort.

FIGS. 7A-7E show plots and tables of fibrosis scores and asparagine and tryptophan metabolism in mice following bacterial inoculation or vehicle treatment. FIG. 7A is a plot of stool levels of asparagine in mice without bacterial inoculation or inoculated with the bacterial consortium. FIG. 7B is a table of Spearman's correlations between stool levels of asparagine and liver fibrosis scores. FIG. 7C shows a series of plots of hepatic levels of tryptophan, kynurenine and tryptophan-to-kynurenine ratio in mice. FIG. 7D is a table of Spearman's correlations between hepatic levels of tryptophan, kynurenine and the tryptophan-to-kynurenine ratio and liver fibrosis scores. FIG. 7E is a table of Spearman's correlation between hepatic levels of tryptophan, kynurenine and the tryptophan-to-kynurenine ratio and total OTU sequence reads in the liver.

FIG. 8 is a plot that shows the contribution of various factors to variation in the fibrosis-associated microbiome signature.

### DETAILED DESCRIPTION

Before the present bacterial consortias, compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present disclosure is based on the seminal discovery that Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis are therapeutic and hepatoprotective. Increasing an abundance of one or more of these species in a gut microbiome protects against a myriad of liver diseases, including steatosis, nonalcoholic steatohepatitis (NASH), nonalcoholic fatty liver disease (NAFLD), liver fibrosis, and cirrhosis of the liver, as well as common comorbidities thereof. For example, as demonstrated in EXAMPLE 3, administration of one or more of these bacteria can inhibit liver fibrosis progression in subjects with NAFLD and NASH.

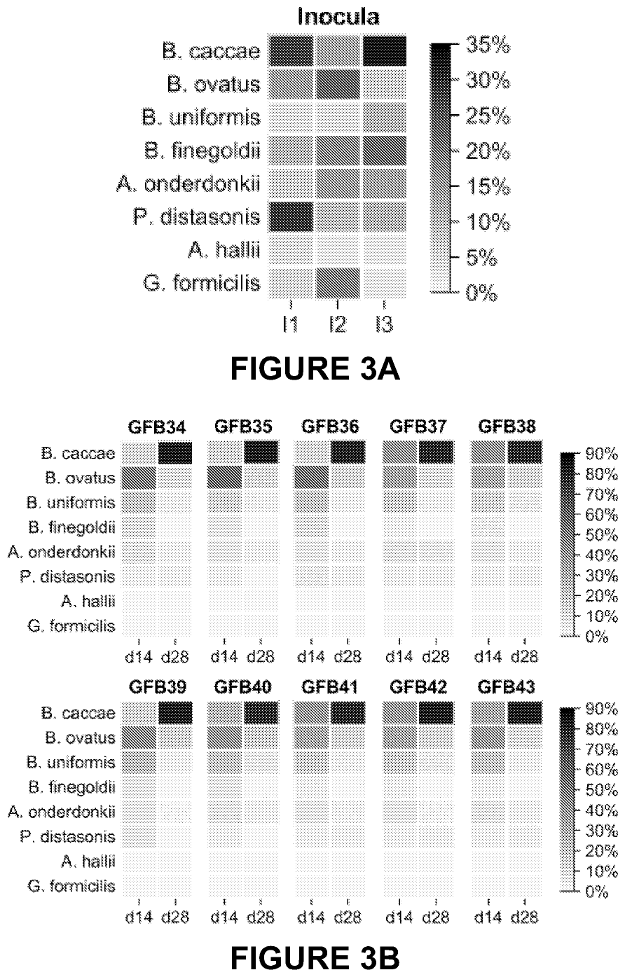

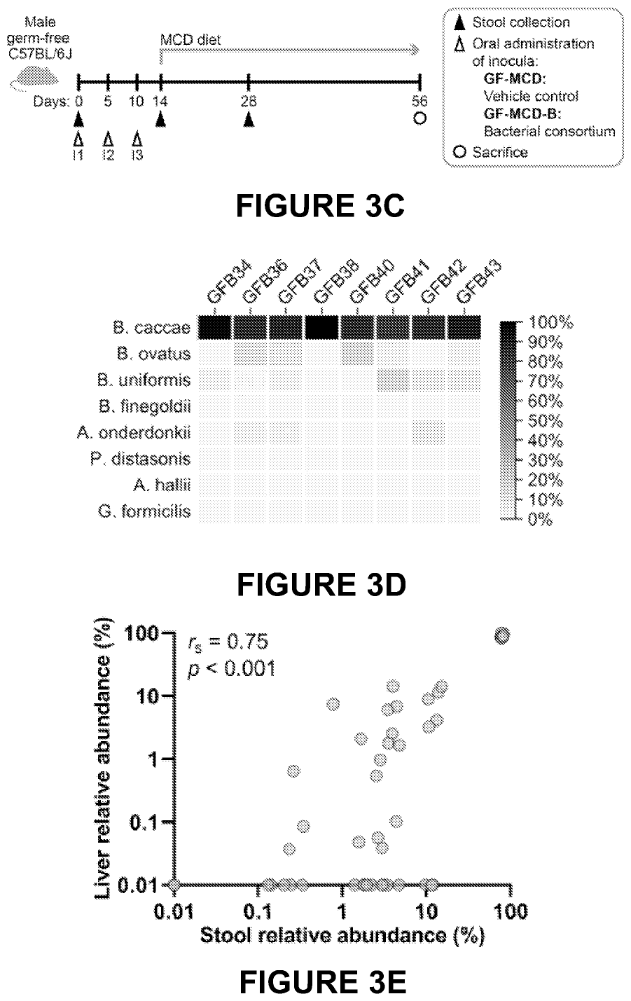

Aspects of the present disclosure provide a composition including Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, or a combination thereof. These bacteria are not only capable of colonizing the human digestive tract but can stably persist in a mammalian gut for months following initial administration (for example as demonstrated in FIGS. 3A-3B of EXAMPLE 3), such that regimens of these bacteria can affect short and long-term changes in gut microbial consortia. As detailed herein, these bacteria can be hepatoprotective and therapeutic. Without being bound by theory, it is hypothesized that administration of these bacteria can increase their abundance within a gut microbiome, which in turn can promote liver health, inhibit liver deterioration, and may decrease the abundance of hepatically deleterious microbiota, thereby diminishing gut microbiome-mediated liver damage and stress. For example, a surprising discovery disclosed herein is that these eight bacteria synergistically modify gut amino acid compositions in a manner that promotes liver health.

Further disclosed herein are methods of treating a disease or condition by administering a composition of the present disclosure to a subject in need thereof. In some aspects, the disease or condition is steatosis, nonalcoholic steatohepatitis (NASH), nonalcoholic fatty liver disease (NAFLD), liver fibrosis, cirrhosis of the liver, or a combination thereof. In some of the methods disclosed herein, the subject is administered the composition after being identified as having low intestinal levels of one or more of the bacteria disclosed herein.

### Compositions

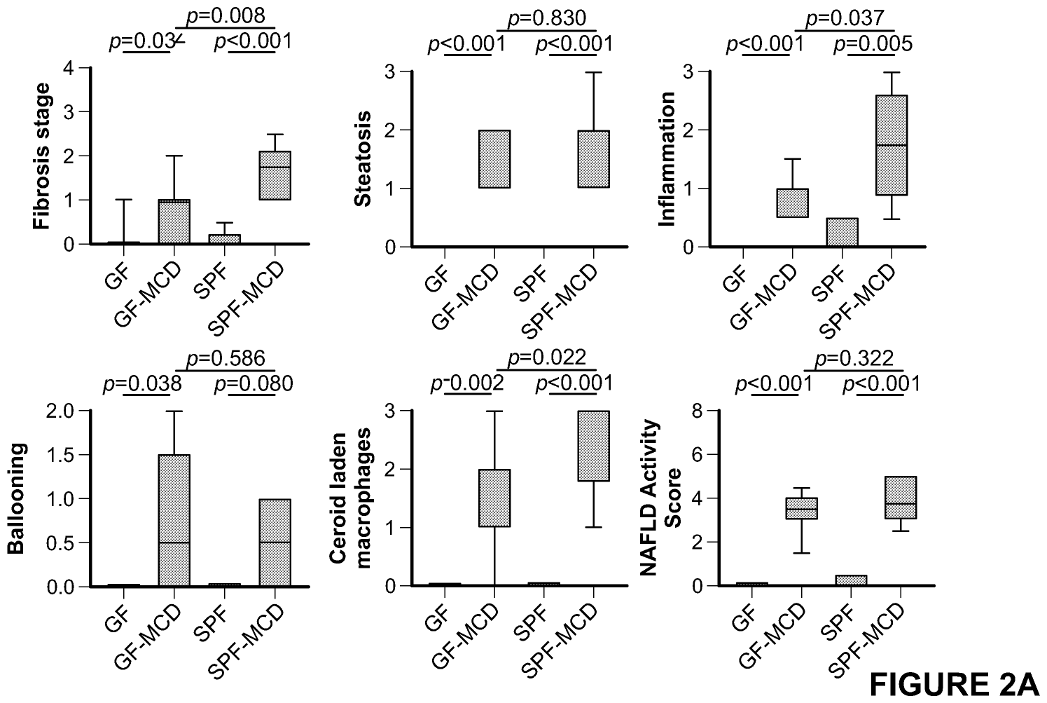

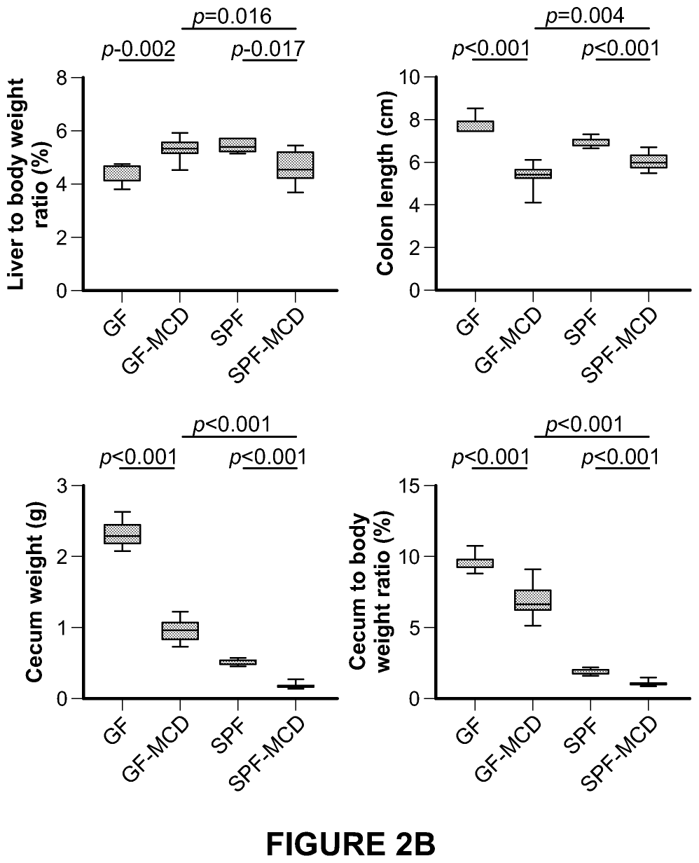

In some aspects, the composition includes a plurality of bacteria selected from the group: Bacteroides finegoldii, Bacteroides caccae, Bacteroides ovatus, Bacteroides umformis, Parabacteroides distasonis, Alistipes onderdonkii, Anaerobutyricum hallii, Gemmiger formicilis, and mutants thereof. As demonstrated herein (for example in FIGS. 2-4 of EXAMPLE 3), a combination of bacterial species disclosed herein can provide synergistic effects for liver protection and health beyond those expected from the species taken individually. Leveraging this discovery, in some aspects, the composition includes at least two of the bacteria or mutants thereof. In some aspects, the composition includes at least three of the bacteria or mutants thereof. In some aspects, the composition includes at least four of the bacteria or mutants thereof. In some aspects, the composition includes at least five of the bacteria or mutants thereof. In some aspects, the composition includes at least six of the bacteria or mutants thereof. In some aspects, the composition includes at least seven of the bacteria or mutants thereof. In some aspects, the composition includes all eight of the bacteria or mutants thereof. In some aspects, the bacteria are alive.

In some aspects, the composition includes a functional mutant of Bacteroides finegoldii, a functional mutant of Bacteroides caccae, a functional mutant of Bacteroides ovatus, a functional mutant of Bacteroides uniformis, a functional mutant of Parabacteroides distasonis, a functional mutant of Alistipes onderdonkii, a functional mutant of Anaerobutyricum hallii, a functional mutant of Gemmiger formicilis, or a combination thereof. As used herein, the term “functional mutant” can denote a strain which has similar probiotic activity as a wild-type strain of the same species. For example, a functional mutant of Bacteroides finegoldii can have similar probiotic activity as strain DSM 17565 deposited with the DSMZ.

The composition can primarily include probiotic bacteria. For example, in some aspects, at least 50% of bacteria of the composition (e.g., total count as determined by relative numbers of reads of 16S sequences) are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, at least 60% of bacteria of the composition are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, at least 70% of bacteria of the composition are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, at least 80% of bacteria of the composition are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, at least 90% of bacteria of the composition are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, at least 95% of bacteria of the composition are Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis, a mutant thereof, or a combination thereof. In some aspects, the composition is free of fungal microbiota, including abundant human gut microflora such as Candida albicans.

In some aspects, the composition includes a plurality of bacteria selected from the group: Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis. In some aspects, the composition includes at least two of the bacteria. In some aspects, the composition includes at least three of the bacteria. In some aspects, the composition includes at least four of the bacteria. In some aspects, the composition includes at least five of the bacteria. In some aspects, the composition includes at least six of the bacteria. In some aspects, the composition includes at least seven of the bacteria. In some aspects, the composition includes all eight of the bacteria.

Relative abundances of bacteria of the composition can be adjusted as determined appropriate for a subject. For example, a bacterium can be provided at less than about 1% relative abundance, less than about 2%, less than 3% etc. to less than 100%. For example, relative abundance may be between 0% and 100%, between 1% and 100%, between 2% and 100% relative abundance within a composition and so on. In certain illustrative examples herein, the relative abundance is between about 1% and 90%.

In some aspects, a relative abundance of Bacteroides finegoldii among bacteria of the composition (e.g., as determined by 16S profiling) is between about 1% and 90%. In some aspects, a relative abundance of Bacteroides finegoldii among bacteria of the composition is between about 4% and 60%. In some aspects, a relative abundance of Bacteroides finegoldii among bacteria of the composition is between about 6% and 45%. In some aspects, a relative abundance of Bacteroides finegoldii among bacteria of the composition is between about 8% and 30%. In some aspects, a relative abundance of Bacteroides finegoldii among bacteria of the composition is between about 12% and 20%.

In some aspects, a relative abundance of Bacteroides caccae among bacteria of the composition is between about 1% and 90%. In some aspects, a relative abundance of Bacteroides caccae among bacteria of the composition is between about 4% and 75%. In some aspects, a relative abundance of Bacteroides caccae among bacteria of the composition is between about 8% and 60%. In some aspects, a relative abundance of Bacteroides caccae among bacteria of the composition is between about 11% and 45%. In some aspects, a relative abundance of Bacteroides caccae among bacteria of the composition is between about 14% and 26%.

In some aspects, a relative abundance of Bacteroides ovatus among bacteria of the composition is between about 1% and 90%. In some aspects, a relative abundance of Bacteroides ovatus among bacteria of the composition is between about 3% and 55%. In some aspects, a relative abundance of Bacteroides ovatus among bacteria of the composition is between about 5% and 40%. In some aspects, a relative abundance of Bacteroides ovatus among bacteria of the composition is between about 7% and 27%. In some aspects, a relative abundance of Bacteroides ovatus among bacteria of the composition is between about 9% and 20%. In some aspects, a relative abundance of Bacteroides ovatus among bacteria of the composition is between about 11% and 17%.

In some aspects, a relative abundance of Bacteroides uniformis among bacteria of the composition is between about 1% and 90%. In some aspects, a relative abundance of Bacteroides uniformis among bacteria of the composition is between about 1% and 25%. In some aspects, a relative abundance of Bacteroides uniformis among bacteria of the composition is between about 2% and 20%. In some aspects, a relative abundance of Bacteroides umformis among bacteria of the composition is between about 2.5% and 12.5%. In some aspects, a relative abundance of Bacteroides umformis among bacteria of the composition is between about 3% and 10%.

In some aspects, a relative abundance of Parabacteroides distasonis among bacteria of the composition is between about 1% and 90%. In some aspects, a relative abundance of Parabacteroides distasonis among bacteria of the composition is between about 4% and 60%. In some aspects, a relative abundance of Parabacteroides distasonis among bacteria of the composition is between about 6% and 40%. In some aspects, a relative abundance of Parabacteroides distasonis among bacteria of the composition is between about 8% and 31%. In some aspects, a relative abundance of Parabacteroides distasonis among bacteria of the composition is between about 10% and 20%.

In some aspects, a relative abundance of Alistipes onderdonkii among bacteria of the composition is between about 1% and 90%. In some aspects, a relative abundance of Alistipes onderdonkii among bacteria of the composition is between about 2% and 50%. In some aspects, a relative abundance of Alistipes onderdonkii among bacteria of the composition is between about 4% and 32%. In some aspects, a relative abundance of Alistipes onderdonkii among bacteria of the composition is between about 5% and 21%. In some aspects, a relative abundance of Alistipes onderdonkii among bacteria of the composition is between about 6% and 15%.

In some aspects, a relative abundance of Anaerobutyricum hallii among the bacteria of the composition is between about 1% and 90%. In some aspects, a relative abundance of Anaerobutyricum hallii among the bacteria of the composition is between about 0.5% and 20%. In some aspects, a relative abundance of Anaerobutyricum hallii among the bacteria of the composition is between about 1% and 12%. In some aspects, a relative abundance of Anaerobutyricum hallii among the bacteria of the composition is between about 1% and 6%. In some aspects, a relative abundance of Anaerobutyricum hallii among the bacteria of the composition is between about 1.5% and 8%. In some aspects, a relative abundance of Anaerobutyricum hallii among the bacteria of the composition is between about 1.5% and 10%.

In some aspects, a relative abundance of Gemmiger formicilis among the bacteria of the composition is between about 1% and 90%. In some aspects, a relative abundance of Gemmiger formicilis among the bacteria of the composition is between about 2% and 35%. In some aspects, a relative abundance of Gemmiger formicilis among the bacteria of the composition is between about 3% and 25%. In some aspects, a relative abundance of Gemmiger formicilis among the bacteria of the composition is between about 4% and 16%. In some aspects, a relative abundance of Gemmiger formicilis among the bacteria of the composition is between about 6% and 12%.

A composition of the present disclosure can include a high proportion of Gram-negative bacteria. As gut microbial Gram-negative bacteria can promote NAFLD progression, for example by affecting proinflammatory TLR4 signaling, a surprising discovery presented herein is that some Gram-negative bacteria can elicit hepatoprotective responses when administered to either healthy or NAFLD-afflicted subjects. Without being bound by theory, it is hypothesized herein that a composition of the present disclosure may promote liver health by increasing the proportion of anti-inflammatory or immunoinhibitory Gram-negative bacteria within a digestive tract, wherein certain Bacteroides species may produce TLR4-suppressive lipopolysaccharides with underacylated lipid A structures. Among the bacteria disclosed herein, Bacteroides finegoldii, Bacteroides caccae, Bacteroides ovatus, Bacteroides uniformis, Parabacteroides distasonis, and Alistipes onderdonkii are Gram-negative, while Gemmiger formicilis is Gram-negative to Gram-variable. Following from this discovery, in some aspects, at least about 50% of bacteria of a composition are Gram-negative bacteria. In some aspects, at least about 60% of bacteria of a composition are Gram-negative bacteria. In some aspects, at least about 70% of bacteria of a composition are Gram-negative bacteria. In some aspects, at least about 80% of bacteria of a composition are Gram-negative bacteria. In some aspects, at least about 90% of bacteria of a composition are Gram-negative bacteria. In some aspects, the Gram-negative bacteria are one or more of Bacteroides finegoldii, Bacteroides caccae, Bacteroides ovatus, Bacteroides uniformis, Parabacteroides distasonis, Alistipes onderdonkii, and mutants thereof.

In some aspects, the composition includes a bacterium of the genus Bacteroides. In some aspects, the composition includes at least two bacteria of the genus Bacteroides. In some aspects, the composition includes at least three bacteria of the genus Bacteroides. In some aspects, the composition includes at least four bacteria of the genus Bacteroides. In some aspects, at least 50% of bacteria of the composition are Bacteroides (e.g., total count as determined by relative numbers of reads of 16S sequences). In some aspects, at least 60% of bacteria of the composition are Bacteroides. In some aspects, at least 70% of bacteria of the composition are Bacteroides. In some aspects, at least 80% of bacteria of the composition are Bacteroides. In some aspects, at least 90% of bacteria of the composition are Bacteroides.

In some aspects, the composition includes a bacterium of the genus Bacteroides and a bacterium of the genus Parabacteroides. In some aspects, the composition includes a bacterium of the genus Bacteroides and a bacterium of the genus Alistipes. In some aspects, the composition includes a bacterium of the genus Bacteroides and a bacterium of the genus Anaerobutyricum. In some aspects, the composition includes a bacterium of the genus Bacteroides and a bacterium of the genus Alistipes. In some aspects, the composition includes a bacterium of the genus Bacteroides and a bacterium of the genus Gemmiger. In some aspects, the composition includes a bacterium of the genus Bacteroides, a bacterium of the genus Parabacteroides, and a bacterium of the genus Alistipes. In some aspects, the composition includes a bacterium of the genus Bacteroides, a bacterium of the genus Parabacteroides, and a bacterium of the genus Anaerobutyricum. In some aspects, the composition includes a bacterium of the genus Bacteroides, a bacterium of the genus Parabacteroides, and a bacterium of the genus Gemmiger. In some aspects, the composition includes a bacterium of the genus Bacteroides, a bacterium of the genus Anaerobutyricum, and a bacterium of the genus Gemmiger. In some aspects, the composition includes a bacterium of the genus Bacteroides, a bacterium of the genus Alistipes, and a bacterium of the genus Gemmiger. In some aspects, the composition includes a bacterium of the genus Bacteroides, a bacterium of the genus Anaerobutyricum, and a bacterium of the genus Gemmiger. In some aspects, the composition includes a bacterium of the genus Bacteroides, a bacterium of the genus Parabacteroides, a bacterium of the genus Alistipes, and a bacterium of the genus Anaerobutyricum. In some aspects, the composition includes a bacterium of the genus Bacteroides, a bacterium of the genus Parabacteroides, a bacterium of the genus Alistipes, and a bacterium of the genus Gemmiger. In some aspects, the composition includes a bacterium of the genus Bacteroides, a bacterium of the genus Parabacteroides, a bacterium of the genus Anaerobutyricum, and a bacterium of the genus Gemmiger. In some aspects, the composition includes a bacterium of the genus Bacteroides, a bacterium of the genus Alistipes, a bacterium of the genus Anaerobutyricum, and a bacterium of the genus Gemmiger. In some aspects, the composition includes a bacterium of the genus Bacteroides, a bacterium of the genus Parabacteroides, a bacterium of the genus Alistipes, a bacterium of the genus Anaerobutyricum, and a bacterium of the genus Gemmiger. In some aspects, the Bacteroides is Bacteroides finegoldii, Bacteroides caccae, Bacteroides ovatus, Bacteroides uniformis, a mutant thereof, or a combination thereof (e.g., a mutant strain of Bacteroides finegoldii and a mutant strain of Bacteroides caccae, or two mutant strains of Bacteroides uniformis). In some aspects, the Parabacteroides is Parabacteroides distasonis or a mutant thereof. In some aspects, the Alistipes is Alistipes onderdonkii or a mutant thereof. In some aspects, the Anaerobutyricum hallii is Anaerobutyricum hallii or a mutant thereof. In some aspects, the Gemmiger is Gemmiger formicilis or a mutant thereof.

In some aspects, the composition includes between about 10⁶ and 10¹², between about 10⁶ and 10¹¹, between about 10⁶ and 10¹⁰, between about 10⁶ and 10⁹, between about 10⁶ and 10⁸, between about 10⁶ and 10⁷, between about 10⁷ and 10¹², between about 10⁷ and 10¹¹, between about 10⁷ and 10¹⁰, between about 10⁷ and 10⁹, between about 10⁷ and 10⁸, between about 10⁸ and 10¹², between about 10⁸ and 10¹⁰, between about 10⁸ and 10¹⁰, between about 10⁸ and 10⁹, between about 10⁹ and 10¹², between about 10⁹ and 10¹¹, between about 10⁹ and 10¹⁰, between about 10¹⁰ and 10¹², between about 10¹⁰ and 10¹¹, or between about 10¹¹ and 10¹² CFU of live bacteria. aspectsaspectsaspectsaspects

A composition of the present disclosure can be formulated for oral administration. Non-limiting examples of such formulations include beverages, syrups, caplets, capsules (e.g., liquid-gel capsules or gelatin capsules), lozenges, gels, colloids, solid bars, spreads (e.g., nut butter-based spreads), pills, powders, or combinations thereof.

The composition can also be formulated for rectal administration, for example as a solid or cream-based suppository. Suppository formulations are well known in the art, and include those described in Remington's Pharmaceutical Sciences, 18th Edition (1990). A suppository can be formulated with a non-irritating excipient or carrier such as cocoa butter, a hard wax, an anhydrous fat, or a macroglyceride, configuring the composition for stability as a solid below body temperature and to melt or disperse once place within the rectal cavity.

The composition can be formulated with a pharmaceutically acceptable carrier, excipient, or stabilizer. Examples of such ingredients are described in detail in Remington's Pharmaceutical Sciences, 18th Edition (1990). Pharmaceutically acceptable carriers, excipients, or stabilizers may include buffers such as phosphate, acetate, and maleate; antioxidants such as ascorbic acid, ascorbyl palmitate, citric acid, methionine, tartaric acid, and vitamin E; preservatives such as octadecyldimethylbenzyl ammonium chloride, hexamethonium chloride, benzalkonium chloride, benzethonium chloride, and phenol; alkyl parabens such as methyl and propyl paraben; resorcinol; low molecular weight polypeptides (e.g., peptides with about 10 or fewer amino acid residues); proteins such as serum albumin and collagen; amino acids such as glycine, glutamate, and lysine; monosaccharides, disaccharides, and other carbohydrates including glucose, mannose, or dextrins; chelating agents such as EDTA; hydrophilic polymers such as polyvinylpyrrolidone and polyethylene glycol; sugars such as sucrose, mannitol, galactose, and trehalose; and/or non-ionic surfactants such as glyceryl monostearate and sorbitan monopalmitate. Examples of pharmaceutically acceptable carriers include, but are not limited to ointments, micellular compositions, colloids, creams, gels, and emulsions. Examples of diluents include, but are not limited to, water, vegetable oils (such as soybean, sunflower, and palm oils), animal fats (such as fish oil and tallow), milk fats, and organic solvents (such as dimethyl sulfoxide).

### Bacterial Strains

Disclosed herein are numerous bacterial species which can promote liver health and protect against liver fibrosis and disease progression. While prevailing models suggest that LPS may hasten NAFLD and NASH development, Gram-positive and Gram-negative species spanning the genera Bacteroides, Parabacteroides, Alistipes, Anaerobutyricum, and Gemmiger were identified as hepatoprotective and therapeutic. While Bacteroides are prevalent in human digestive tracts, Parabacteroides, Alistipes, Anaerobutyricum, and Gemmiger are typically of low relatively abundances.

### Bacteroides

A composition of the present disclosure can include one or more species of the genus Bacteroides, a major commensal genus in the human gut. As members of Bacteroides are gram-negative and therefore major producers of lipopolysaccharides (LPS), well known agonists of proinflammatory TLR4 signaling pathways, the identification of certain Bacteroides species as hepatoprotective was unexpected. To this point, correlations between Bacteroides abundance and liver damage has been observed in certain populations, for example in patients with NASH and fibrosis stage F2+ (Boursier et al. J Hepatol, 2016; 65(3):570-8). Without being bound by theory, the Bacteroides species disclosed herein may produce immunoinhibitory forms of LPS with underacylated lipid A structures, and therefore may suppress TLR4 activation and associated proinflammatory signaling.

In some aspects, Bacteroides of a composition of the present disclosure includes Bacteroides finegoldii or a mutant thereof, Bacteroides caccae or a mutant thereof, Bacteroides ovatus or a mutant thereof, Bacteroides uniformis or a mutant thereof, or a combination thereof. In some aspects, Bacteroides of a composition of the present disclosure includes at least two of Bacteroides finegoldii or a mutant thereof, Bacteroides caccae or a mutant thereof, Bacteroides ovatus or a mutant thereof, and Bacteroides umformis or a mutant thereof. In some aspects, Bacteroides of a composition of the present disclosure includes at least three of Bacteroides finegoldii or a mutant thereof, Bacteroides caccae or a mutant thereof, Bacteroides ovatus or a mutant thereof, and Bacteroides uniformis or a mutant thereof. In some aspects, Bacteroides of a composition of the present disclosure includes Bacteroides finegoldii or a mutant thereof, Bacteroides caccae or a mutant thereof, Bacteroides ovatus or a mutant thereof, and Bacteroides uniformis or a mutant thereof.

In some aspects, Bacteroides finegoldii of a composition of the present disclosure has at least 90% identity to the nucleotide sequence of strain DSM 17565 deposited with the Deutsche Sammlung von Mikroorganismen und Zellkulturen (DSMZ, Germany). For example, Bacteroides finegoldii of a composition of the present disclosure can be a mutant strain which has 90.5% identity to the nucleotide sequence of strain DSM 17565 deposited with the DSMZ. In some aspects, the Bacteroides finegoldii has at least 91% identity to the nucleotide sequence of strain DSM 17565 deposited with the DMSZ. In some aspects, the Bacteroides finegoldii has at least 92% identity to the nucleotide sequence of strain DSM 17565 deposited with the DMSZ. In some aspects, the Bacteroides finegoldii has at least 93% identity to the nucleotide sequence of strain DSM 17565 deposited with the DMSZ. In some aspects, the Bacteroides finegoldii has at least 94% identity to the nucleotide sequence of strain DSM 17565 deposited with the DMSZ. In some aspects, the Bacteroides finegoldii has at least 95% identity to the nucleotide sequence of strain DSM 17565 deposited with the DMSZ. In some aspects, the Bacteroides finegoldii has at least 96% identity to the nucleotide sequence of strain DSM 17565 deposited with the DMSZ. In some aspects, the Bacteroides finegoldii has at least 97% identity to the nucleotide sequence of strain DSM 17565 deposited with the DMSZ. In some aspects, the Bacteroides finegoldii has at least 98% identity to the nucleotide sequence of strain DSM 17565 deposited with the DMSZ. In some aspects, the Bacteroides finegoldii has at least 99% identity to the nucleotide sequence of strain DSM 17565 deposited with the DSMZ.

In some aspects, Bacteroides caccae of a composition of the present disclosure has at least 90% identity to the nucleotide sequence of strain DSM 19024 deposited with the DMSZ. In some aspects, the Bacteroides caccae has at least 91% identity to the nucleotide sequence of strain DSM 19024 deposited with the DMSZ. In some aspects, the Bacteroides caccae has at least 92% identity to the nucleotide sequence of strain DSM 19024 deposited with the DMSZ. In some aspects, the Bacteroides caccae has at least 93% identity to the nucleotide sequence of strain DSM 19024 deposited with the DMSZ. In some aspects, the Bacteroides caccae has at least 94% identity to the nucleotide sequence of strain DSM 19024 deposited with the DMSZ. In some aspects, the Bacteroides caccae has at least 95% identity to the nucleotide sequence of strain DSM 19024 deposited with the DMSZ. In some aspects, the Bacteroides caccae has at least 96% identity to the nucleotide sequence of strain DSM 19024 deposited with the DMSZ. In some aspects, the Bacteroides caccae has at least 97% identity to the nucleotide sequence of strain DSM 19024 deposited with the DMSZ. In some aspects, the Bacteroides caccae has at least 98% identity to the nucleotide sequence of strain DSM 19024 deposited with the DMSZ. In some aspects, the Bacteroides caccae has at least 99% identity to the nucleotide sequence of strain DSM 19024 deposited with the DMSZ.

In some aspects, Bacteroides ovatus of a composition of the present disclosure has at least 90% identity to the nucleotide sequence of strain DSM 1896 deposited with the DMSZ. In some aspects, the Bacteroides ovatus has at least 91% identity to the nucleotide sequence of strain DSM 1896 deposited with the DMSZ. In some aspects, the Bacteroides ovatus has at least 92% identity to the nucleotide sequence of strain DSM 1896 deposited with the DMSZ. In some aspects, the Bacteroides ovatus has at least 93% identity to the nucleotide sequence of strain DSM 1896 deposited with the DMSZ. In some aspects, the Bacteroides ovatus has at least 94% identity to the nucleotide sequence of strain DSM 1896 deposited with the DMSZ. In some aspects, the Bacteroides ovatus has at least 95% identity to the nucleotide sequence of strain DSM 1896 deposited with the DMSZ. In some aspects, the Bacteroides ovatus has at least 96% identity to the nucleotide sequence of strain DSM 1896 deposited with the DMSZ. In some aspects, the Bacteroides ovatus has at least 97% identity to the nucleotide sequence of strain DSM 1896 deposited with the DMSZ. In some aspects, the Bacteroides ovatus has at least 98% identity to the nucleotide sequence of strain DSM 1896 deposited with the DMSZ. In some aspects, the Bacteroides ovatus has at least 99% identity to the nucleotide sequence of strain DSM 1896 deposited with the DMSZ.

In some aspects, Bacteroides uniformis of a composition of the present disclosure has at least 90% identity to the nucleotide sequence of strain DSM 6597 deposited with the DMSZ. In some aspects, the Bacteroides uniformis has at least 91% identity to the nucleotide sequence of strain DSM 6597 deposited with the DMSZ. In some aspects, the Bacteroides umformis has at least 92% identity to the nucleotide sequence of strain DSM 6597 deposited with the DMSZ. In some aspects, the Bacteroides uniformis has at least 93% identity to the nucleotide sequence of strain DSM 6597 deposited with the DMSZ. In some aspects, the Bacteroides umformis has at least 94% identity to the nucleotide sequence of strain DSM 6597 deposited with the DMSZ. In some aspects, the Bacteroides uniformis has at least 95% identity to the nucleotide sequence of strain DSM 6597 deposited with the DMSZ. In some aspects, the Bacteroides umformis has at least 96% identity to the nucleotide sequence of strain DSM 6597 deposited with the DMSZ. In some aspects, the Bacteroides uniformis has at least 97% identity to the nucleotide sequence of strain DSM 6597 deposited with the DMSZ. In some aspects, the Bacteroides umformis has at least 98% identity to the nucleotide sequence of strain DSM 6597 deposited with the DMSZ. In some aspects, the Bacteroides uniformis has at least 99% identity to the nucleotide sequence of strain DSM 6597 deposited with the DMSZ.

### Parabacteroides

A composition can include one or more species of the genus Parabacteroides.

Parabacteroides are gram-negative, non-spore-forming, obligate anaerobic bacteria present in the digestive tracts of some animals. At present, there is little consensus as to whether particular species of Parabacteroides are commensal or pathogenic. For example, there are conflicting reports concerning the role of Parabacteroides distasonis in inflammatory bowel disease (IBD), diabetes, and autoimmune disorders (Ezeji et al. Gut Microbes, 2021; 13). However, it was identified herein that Parabacteroides distasonis can inhibit the progression of steatosis, nonalcoholic steatohepatitis, nonalcoholic fatty liver disease, and liver fibrosis.

In some aspects, a species of Parabacteroides in a composition disclosed herein is Parabacteroides distasonis. In some aspects, Parabacteroides distasonis has at least 90% identity to the nucleotide sequence of strain DSM 20701 deposited with the DMSZ. In some aspects, the Parabacteroides distasonis has at least 91% identity to the nucleotide sequence of strain DSM 20701 deposited with the DMSZ. In some aspects, the Parabacteroides distasonis has at least 92% identity to the nucleotide sequence of strain DSM 20701 deposited with the DMSZ. In some aspects, the Parabacteroides distasonis has at least 93% identity to the nucleotide sequence of strain DSM 20701 deposited with the DMSZ. In some aspects, the Parabacteroides distasonis has at least 94% identity to the nucleotide sequence of strain DSM 20701 deposited with the DMSZ. In some aspects, the Parabacteroides distasonis has at least 95% identity to the nucleotide sequence of strain DSM 20701 deposited with the DMSZ. In some aspects, the Parabacteroides distasonis has at least 96% identity to the nucleotide sequence of strain DSM 20701 deposited with the DMSZ. In some aspects, the Parabacteroides distasonis has at least 97% identity to the nucleotide sequence of strain DSM 20701 deposited with the DMSZ. In some aspects, the Parabacteroides distasonis has at least 98% identity to the nucleotide sequence of strain DSM 20701 deposited with the DMSZ. In some aspects, the Parabacteroides distasonis has at least 99% identity to the nucleotide sequence of strain DSM 20701 deposited with the DMSZ.

### Alistipes

A composition can include one or more species of the genus Alistipes. The genus Alistipes encompasses a diverse set of anaerobic bacteria primarily distributed throughout the human digestive tract as well as numerous types of abscesses. Some species of Alistipes have been shown to produce succinate along with minor amounts of acetate and propionate in physiological conditions. While certain species of Alistipes are associated with obesity and anorexia (Parker et al. Front. Immunol., 2020; 11(906)), it was determined herein that Alistipes onderdonkii can be hepatoprotective.

In some aspects, a species of Alistipes in a composition disclosed herein is Alistipes onderdonkii. In some aspects, the Alistipes onderdonkii has at least 90% sequence identity to the nucleotide sequence of strain DSM 32839 deposited with the DSMZ. In some aspects, the Alistipes onderdonkii has at least 91% sequence identity to the nucleotide sequence of strain DSM 32839 deposited with the DSMZ. In some aspects, the Alistipes onderdonkii has at least 92% sequence identity to the nucleotide sequence of strain DSM 32839 deposited with the DSMZ. In some aspects, the Alistipes onderdonkii has at least 93% sequence identity to the nucleotide sequence of strain DSM 32839 deposited with the DSMZ. In some aspects, the Alistipes onderdonkii has at least 94% sequence identity to the nucleotide sequence of strain DSM 32839 deposited with the DSMZ. In some aspects, the Alistipes onderdonkii has at least 95% sequence identity to the nucleotide sequence of strain DSM 32839 deposited with the DSMZ. In some aspects, the Alistipes onderdonkii has at least 96% sequence identity to the nucleotide sequence of strain DSM 32839 deposited with the DSMZ. In some aspects, the Alistipes onderdonkii has at least 97% sequence identity to the nucleotide sequence of strain DSM 32839 deposited with the DSMZ. In some aspects, the Alistipes onderdonkii has at least 98% sequence identity to the nucleotide sequence of strain DSM 32839 deposited with the DSMZ. In some aspects, the Alistipes onderdonkii has at least 99% sequence identity to the nucleotide sequence of strain DSM 32839 deposited with the DSMZ.

### Anaerobutyricum

A composition can include one or more species of the genus Anaerobutyricum. Of this genus, A. hallii is one of few known strains that can utilize lactate for the production of butyrate and can convert primary bile acids to secondary bile acids (Engels et al. Frontiers in Microbiology, 2016; 7:713). In some aspects, a species of Anaerobutyricum in a composition disclosed herein is Anaerobutyricum hallii. In some aspects, the Anaerobutyricum hallii has at least 90% sequence identity to the nucleotide sequence of strain DSM 3353 deposited with the DSMZ. In some aspects, the Anaerobutyricum hallii has at least 91% sequence identity to the nucleotide sequence of strain DSM 3353 deposited with the DSMZ. In some aspects, the Anaerobutyricum hallii has at least 92% sequence identity to the nucleotide sequence of strain DSM 3353 deposited with the DSMZ. In some aspects, the Anaerobutyricum hallii has at least 93% sequence identity to the nucleotide sequence of strain DSM 3353 deposited with the DSMZ. In some aspects, the Anaerobutyricum hallii has at least 94% sequence identity to the nucleotide sequence of strain DSM 3353 deposited with the DSMZ. In some aspects, the Anaerobutyricum hallii has at least 95% sequence identity to the nucleotide sequence of strain DSM 3353 deposited with the DSMZ. In some aspects, the Anaerobutyricum hallii has at least 96% sequence identity to the nucleotide sequence of strain DSM 3353 deposited with the DSMZ. In some aspects, the Anaerobutyricum hallii has at least 97% sequence identity to the nucleotide sequence of strain DSM 3353 deposited with the DSMZ. In some aspects, the Anaerobutyricum hallii has at least 98% sequence identity to the nucleotide sequence of strain DSM 3353 deposited with the DSMZ. In some aspects, the Anaerobutyricum hallii has at least 99% sequence identity to the nucleotide sequence of strain DSM 3353 deposited with the DSMZ.

### Gemmiger

A composition can include one or more species of Gemmiger, a genus which encompasses a number of Gram-negative and Gram-variable obligate anaerobic species. In some aspects, a species of Gemmiger in a composition disclosed herein is Gemmiger formicilis. Gemmiger formicilis has been shown to decrease following vitamin C administration (Otten et al. Antioxidants, 2021; 10:1278), and may promote immune-related colitis (Martin et al. JHEP Reports, 2020; 2(6):100170). However, as determined herein, Gemmiger formicilis can also inhibit NAFLD and promote liver health.

In some aspects, the Gemmiger formicilis has at least 90% sequence identity to the nucleotide sequence of strain ATCC 27749 deposited with the American Type Culture Collection (ATCC). In some aspects, the Gemmiger formicilis has at least 91% sequence identity to the nucleotide sequence of strain ATCC 27749 deposited with the ATCC. In some aspects, the Gemmiger formicilis has at least 92% sequence identity to the nucleotide sequence of strain ATCC 27749 deposited with the ATCC. In some aspects, the Gemmiger formicilis has at least 93% sequence identity to the nucleotide sequence of strain ATCC 27749 deposited with the ATCC. In some aspects, the Gemmiger formicilis has at least 94% sequence identity to the nucleotide sequence of strain ATCC 27749 deposited with the ATCC. In some aspects, the Gemmiger formicilis has at least 95% sequence identity to the nucleotide sequence of strain ATCC 27749 deposited with the ATCC. In some aspects, the Gemmiger formicilis has at least 96% sequence identity to the nucleotide sequence of strain ATCC 27749 deposited with the ATCC. In some aspects, the Gemmiger formicilis has at least 97% sequence identity to the nucleotide sequence of strain ATCC 27749 deposited with the ATCC. In some aspects, the Gemmiger formicilis has at least 98% sequence identity to the nucleotide sequence of strain ATCC 27749 deposited with the ATCC. In some aspects, the Gemmiger formicilis has at least 99% sequence identity to the nucleotide sequence of strain ATCC 27749 deposited with the ATCC.

Aspects of the present disclosure provide methods for treating subjects by administering a composition of the present disclosure. As demonstrated herein, Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, and mutants thereof can be hepatoprotective, for example slowing the progression of steatosis and liver fibrosis. Leveraging this discovery, the present disclosure provides methods of treating a disease or condition in a subject in need thereof by administering a bacterial composition of the present disclosure. In many aspects, the disease or condition is a disease of the liver, such as steatosis, nonalcoholic steatohepatitis (NASH), nonalcoholic fatty liver disease (NAFLD), liver fibrosis, cirrhosis of the liver, or a combination thereof. In some aspects, the subject has NAFLD, NASH, or a combination thereof. In some aspects, administration of the composition inhibits liver fibrosis.

In some embodiments, the present disclosure provides methods of preventing a disease or condition in a subject in need thereof by administering a bacterial composition of the present disclosure. In some aspects, the disease or condition is a disease or condition of the liver. In some aspects, the disease or condition is steatosis, nonalcoholic steatohepatitis (NASH), nonalcoholic fatty liver disease (NAFLD), liver fibrosis, cirrhosis of the liver, or a combination thereof. In some aspects, the subject has NAFLD, NASH, or a combination thereof.

The subject can be administered as a single dose or a plurality of doses of the bacterial composition. In some aspects, the subject is administered a single dose of the bacterial composition. In some aspects, the subject is administered between 2 and 5 doses of the bacterial composition. In some aspects, the subject is administered between 3 and 10 doses of the bacterial composition. In some aspects, the subject is administered the bacterial composition up until alleviation of a disease or condition.

Multiple doses of the composition can be administered at a frequency as determined appropriate for a subject, for example, daily, weekly, monthly and the like. For example, in some aspects, multiple doses of the bacterial composition are administered at a frequency of about once every 1 to 90 days. In some aspects, multiple doses of the bacterial composition are administered at a frequency of once every 3 to 10 days. In some aspects, multiple doses of the bacterial composition are administered at a frequency of once every 5 to 20 days. In some aspects, multiple doses of the bacterial composition are administered at a frequency of once every 10 to 40 days. In some aspects, multiple doses of the bacterial composition are administered at a frequency of once every 25 to 100 days.

In some aspects, a dose of the bacterial composition has between about 10⁶ and 10¹², between about 10⁶ and 10¹¹, between about 10⁶ and 10¹⁰, between about 10⁶ and 10⁹, between about 10⁶ and 10⁸, between about 10⁶ and 10⁷, between about 10⁷ and 10¹², between about 10⁷ and 10¹¹, between about 10⁷ and 10¹⁰, between about 10⁷ and 10⁹, between about 10⁷ and 10⁸, between about 10⁸ and 10¹², between about 10⁸ and 10¹¹, between about 10⁸ and 10¹⁰, between about 10⁸ and 10⁹, between about 10⁹ and 10¹², between about 10⁹ and 10¹¹, between about 10⁹ and 10¹⁰, between about 10¹⁰ and 10¹², between about 10¹⁰ and 10¹¹, or between about 10¹¹ and 10¹² CFU of live bacteria.

In some aspects, the composition is administered orally. Such compositions may be administered as pills, gels, creams, capsules, beverages, foods, gavages, or combinations thereof. The composition can be formulated for delayed release. For example, the composition can be configured to release bacteria within the lower digestive tract, that is, within the jejunum, ileum, or large intestine. To affect delayed release, the composition can be formulated within an enteric coating (i.e., within a medium which is stable within a gastric environment, but which degrades or releases within intestines), or within a solid formulation which degrades within the small or large intestine. The composition can also be configured to release bacteria at a controlled rate, for example within 1 hour, within 6 hours, within 12 hours, within 1 day, within 2 days, or within 3 days of consumption.

In some aspects, the composition is administered rectally, for example as a suppository. The suppository can be formulated for rapid or slow release, for example by modifying the melting temperature or degradation rate of a carrier medium. In some aspects, the suppository is provided as a solid (e.g., a hard wax) or as a cream.

In some aspects, a method of treatment includes diagnostic assessment of an intestinal microbiome of a subject. A representative sample of the subject's intestinal microbiome may be obtained from fecal matter or from a rectal swab from the subject. The sample can then be profiled for a single genus or species or for a plurality of genera and/or species. As it was demonstrated herein that Bacteroides species, Parabacteroides distasonis, Anaerobutyricum hallii, Anaerostipes hadrus, Adlercreutzia equolifaciens and Blautia massiliensis can be depleted in subjects with liver fibrosis, and that Bacteroides finegoldii, Alistipes onderdonkii, Anaerobutyricum hallii and Gemmiger formicilis can be depleted in subjects with advanced liver fibrosis, a method may include measuring an abundance of one or more of these bacteria in an intestinal microbiome of a subject with, suspected of having, or at risk of developing steatosis, NASH, nonalcoholic fatty liver disease (NAFLD), liver fibrosis, cirrhosis of the liver, or a combination of conditions thereof. In some aspects, the method includes determining that a relative abundance of Bacteroides finegoldii is less than about 0.2% in the intestinal microbiome in the subject. In some aspects, the method includes determining that a relative abundance of Bacteroides caccae is less than about 0.1% in the intestinal microbiome in the subject. In some aspects, the method includes determining that a relative abundance of Bacteroides ovatus is less than about 0.5% in the intestinal microbiome in the subject. In some aspects, the method includes determining that a relative abundance of Bacteroides uniformis is less than about 2.0% in the intestinal microbiome in the subject. In some aspects, the method includes determining that a relative abundance of Parabacteroides distasonis is less than about 0.4% in the intestinal microbiome in the subject. In some aspects, the method includes determining that a relative abundance of Alistipes onderdonkii is less than about 0.2% in the intestinal microbiome in the subject. In some aspects, the method includes determining that a relative abundance of Anaerobutyricum hallii is less than about 1.0% in the intestinal microbiome in the subject. In some aspects, the method includes determining that a relative abundance of Gemmiger formicilis is less than about 0.8% in the intestinal microbiome in the subject. In some aspects, the method includes determining the abundances of at least two, at least three, at least four, at least five, at least six, at least seven, or of all eight of of Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis in the intestinal microbiome of the subject.

Relative abundances of bacteria in the intestinal microbiome of the subject are determined during or subsequent to treatment to determine whether the disease or condition was successfully treated in the subject. In such cases, the method can include, for example, determining that a relative abundance of Bacteroides finegoldii is greater than about 0.2% in the intestinal microbiome in the subject, determining that a relative abundance of Bacteroides caccae is greater than about 0.1% in the intestinal microbiome in the subject, determining that a relative abundance of Bacteroides ovatus is greater than about 0.5% in the intestinal microbiome in the subject, determining that a relative abundance of Bacteroides uniformis is greater than about 2.0% in the intestinal microbiome in the subject, determining that a relative abundance of Parabacteroides distasonis is greater than about 0.4% in the intestinal microbiome in the subject, determining that a relative abundance of Alistipes onderdonkii is greater than about 0.2% in the intestinal microbiome in the subject, determining that a relative abundance of Anaerobutyricum hallii is greater than about 1.0% in the intestinal microbiome in the subject, determining that a relative abundance of Gemmiger formicilis is greater than about 0.8% in the intestinal microbiome in the subject, or a combination thereof.

Similarly, the method includes measuring levels of amino acids in stool or liver samples from the subject to determine whether the method of treatment was successful. Accordingly, in some aspects, the method includes measuring less than about 0.05% relative abundance of serine among amino acids in stool of the subject, measuring greater than about 0.005% relative abundance of cysteine among amino acids in stool of the subject, measuring a ratio of cysteine to serine of greater than about 0.2 in stool of the subject, measuring a greater than 0.005% relative abundance among amino acids of homocysteine in liver tissue of the subject, measuring greater than 0.005% relative abundance among amino acids of homocysteine in stool of the subject, measuring greater than 0.05% relative abundance among amino acids of S-adenosylhomocysteine in stool of the subject, measuring greater than 4% relative abundance among amino acids of taurine in stool of the subject, measuring greater than 0.5% relative abundance among amino acids of S-adenosylhomocysteine in liver tissue of the subject, measuring less than 0.1% relative abundance among amino acids of asparagine in stool of the subject, measuring greater than 3% relative abundance among amino acids of tryptophan in stool of the subject, measuring less than 0.15% relative abundance among amino acids of kynurenine in liver tissue of the subject, measuring a kynurenine to tryptophan ratio of less than 0.05 in liver tissue of the subject, or a combination thereof, thereby determining that the disease or condition has been treated in the subject.

The method includes determining whether the treatment was successful by measuring levels of enzymes in stool from the subject. In one aspect, the method includes measuring greater than 10000 copies per million of enzymes from the pentose phosphate pathway non-oxidative branch in the stool of the subject, measuring greater than 12000 copies per million of enzymes from the diacylglycerol biosynthesis I pathway in the stool of the subject, measuring greater than 5000 copies per million of enzymes from the preQ0 biosynthesis pathway in the stool of the subject, measuring greater than 12000 copies per million of enzymes from the CDP-diacylglycerol biosynthesis II pathway in the stool of the subject, or a combination thereof, thereby determining that the disease or condition has been treated in the subject. In another aspect, the method includes measuring greater than 90 copies per million of 2-oxoglutarate in the stool of the subject, measuring greater than 1250 copies per million of cysteine synthase in the stool of the subject, measuring greater than 140 copies per million of penicillin amidase in the stool of the subject, measuring greater than 220 copies per million of N-acetylmuramic acid 6-phosphate etherase in the stool of the subject, measuring greater than 1100 copies per million of 2-C-methyl-D-erythritol 2,4-cyclodiphosphate synthase in the stool of the subject, measuring greater than 400 copies per million of L-rhamnose isomerase in the stool of the subject, or a combination thereof, thereby determining that the disease or condition has been treated in the subject.

In some aspects, the method includes selecting the composition based on the relative abundance(s) of bacteria in the intestinal microbiome of the subject. For example, the method can include administering Bacteroides finegoldii, Bacteroides caccae, Bacteroides ovatus, Bacteroides uniformis, Parabacteroides distasonis, Alistipes onderdonkii, Anaerobutyricum hallii, or Gemmiger formicilis if their abundances within a subjects intestinal microbiome are below desired thresholds (e.g., those outlined above). In some aspects, administration of the bacterial composition is continued until relative abundances of one or more of Bacteroides finegoldii, Bacteroides caccae, Bacteroides ovatus, Bacteroides uniformis, Parabacteroides distasonis, Alistipes onderdonkii, Anaerobutyricum hallii, and Gemmiger formicilis are determined to be above 0.2% for Bacteroides finegoldii, 0.1% for Bacteroides caccae, 0.5% for Bacteroides ovatus, 2.0% for Bacteroides uniformis, 0.4% for Parabacteroides distasonis, 0.2% for Alistipes onderdonkii, 1.0% for Anaerobutyricum hallii, and 0.8% for Gemmiger formicilis, respectively.

In another embodiment, the present disclosure provides a method of identifying a subject as having a condition or being at risk of developing a condition selected from steatosis, nonalcoholic steatohepatitis (NASH), nonalcoholic fatty liver disease (NAFLD), liver fibrosis, or cirrhosis of the liver that includes: i) measuring depletion of Bacteroides finegoldii; Bacteroides caccae; Bacteroides ovatus, Bacteroides uniformis, Parabacteroides distasonis, Alistipes onderdonkii, Anaerobutyricum hallii, Gemmiger formicilis, or a combination thereof in stool of the subject; ii) measuring depletion of a biosynthetic pathway selected from pentose phosphate pathway non-oxidative branch, diacylglycerol biosynthesis I, preQ0 biosynthesis, or CDP-diacylglycerol biosynthesis II in stool of the subject; iii) measuring depletion of 2-oxoglutarate synthase, cysteine synthase, penicillin amidase, N-acetylmuramic acid 6-phosphate etherase, 2-C-methyl-D-erythritol 2,-4-cyclodiphosphate synthase, L-rhamnose isomerase, or a combination thereof in stool of the subject; iv) measuring depletion of cysteine, homocysteine, S-adenosylhomocysteine, tryptophan, or a combination thereof in stool of the subject; v) measuring depletion of homocysteine, S-adenosylhomocysteine, kynurenine, or a combination thereof in liver tissue of the subject; vi) measuring an increase in serine, asparagine, or a combination thereof in stool of the subject; or vii) a combination thereof, thereby identifying the subject as having or being at risk of developing the condition. In particular aspects, the method includes identifying the subject as having the condition. In other aspects, the method includes identifying the subject as being at risk of developing the condition. In particular aspects, the condition is steatosis. In other aspects, the condition is NASH. In further aspects, the condition is NAFLD. In alternative aspects, the condition is liver fibrosis.

In some aspects, the method includes measuring depletion of Bacteroides finegoldii; Bacteroides caccae; Bacteroides ovatus, Bacteroides uniformis, Parabacteroides distasonis, Alistipes onderdonkii, Anaerobutyricum hallii, Gemmiger formicilis, or a combination thereof in stool of the subject. In certain aspects, the method includes measuring depletion of at least two, at least three, at least four, at least five, at least six, at least seven, or eight of Bacteroides finegoldii; Bacteroides caccae; Bacteroides ovatus, Bacteroides uniformis, Parabacteroides distasonis, Alistipes onderdonkii, Anaerobutyricum hallii, Gemmiger formicilis in the stool of the subject. In particular aspects, the method includes determining that a relative abundance of Bacteroides finegoldii is less than about 0.2% in the intestinal microbiome in the subject. In certain aspects, the method includes determining that a relative abundance of Bacteroides caccae is less than about 0.1% in the intestinal microbiome in the subject. In additional aspects, the method includes determining that a relative abundance of Bacteroides ovatus is less than about 0.5% in the intestinal microbiome in the subject. In another aspect, the method includes determining that a relative abundance of Bacteroides uniformis is less than about 2.0% in the intestinal microbiome in the subject. In other aspects, the method includes determining that a relative abundance of Parabacteroides distasonis is less than about 0.4% in the intestinal microbiome in the subject. In some aspects, the method includes determining that a relative abundance of Alistipes onderdonkii is less than about 0.2% in the intestinal microbiome in the subject. In particular aspects, the method includes determining that a relative abundance of Anaerobutyricum hallii is less than about 1.0% in the intestinal microbiome in the subject. In further aspects, the method includes determining that a relative abundance of Gemmiger formicilis is less than about 0.8% in the intestinal microbiome in the subject.

Leveraging the surprising discovery that the expression levels of certain biosynthetic pathways are diminished (relative to healthy subjects) in subjects with liver disease or at risk of developing liver disease, in another aspect, the method includes measuring depletion of a biosynthetic pathway selected from pentose phosphate pathway non-oxidative branch, diacylglycerol biosynthesis I, preQ0 biosynthesis, or CDP-diacylglycerol biosynthesis II in stool of the subject. The method can include measuring depletion of one, two, three, or all four of these pathways. In certain aspects, the method includes measuring less than 10000 copies per million of enzymes from the pentose phosphate pathway non-oxidative branch in the stool of the subject. In further aspects, the method includes measuring less than 9500 copies per million of enzymes from the pentose phosphate pathway non-oxidative branch in the stool of the subject. In other aspects, the method includes measuring less than 12000 copies per million of enzymes from the diacylglycerol biosynthesis I pathway in the stool of the subject. In additional aspects, the method includes measuring less than 11800 copies per million of enzymes from the diacylglycerol biosynthesis I pathway in the stool of the subject. In further aspects, the method includes measuring less than 5000 copies per million of enzymes from the preQ0 biosynthesis pathway in the stool of the subject. In additional aspects, the method includes measuring less than 4800 copies per million of enzymes from the preQ0 biosynthesis pathway in the stool of the subject. In other aspects, the method includes measuring less than 12000 copies per million of enzymes from the CDP-diacylglycerol biosynthesis II pathway in the stool of the subject. In certain aspects, the method includes measuring less than 11800 copies per million of enzymes from the CDP-diacylglycerol biosynthesis II pathway in the stool of the subject.

The method includes measuring depletion of particular enzymes associated with liver disease. For example, in a particular aspect, the method includes measuring depletion of 2-oxoglutarate synthase, cysteine synthase, penicillin amidase, N-acetylmuramic acid 6-phosphate etherase, 2-C-methyl-D-erythritol 2,4-cyclodiphosphate synthase, L-rhamnose isomerase, or a combination thereof in stool of the subject. More specifically, the method can include measuring depletion of one, two, three, four, five, or all six of the aforementioned enzymes. The depletion can be a lower abundance in the stool of the subject than in stool of a healthy subject. In one aspect, the method includes measuring less than 90 copies per million of 2-oxoglutarate in the stool of the subject. In a further aspect, the method includes measuring less than 75 copies per million of 2-oxoglutarate in the stool of the subject. In another aspect, the method includes measuring less than 1250 copies per million of cysteine synthase in the stool of the subject. In a further aspect, the method includes measuring less than 1225 copies per million of cysteine synthase in the stool of the subject. In a further aspect, the method includes measuring less than 140 copies per million of penicillin amidase in the stool of the subject. In another aspect, the method includes measuring less than 80 copies per million of penicillin amidase in the stool of the subject. In an additional aspect, the method includes measuring less than 220 copies per million of N-acetylmuramic acid 6-phosphate etherase in the stool of the subject. In another aspect, the method includes measuring less than 195 copies per million of N-acetylmuramic acid 6-phosphate etherase in the stool of the subject. In another aspect, the method includes measuring less than 1100 copies per million of 2-C-methyl-D-erythritol 2,4-cyclodiphosphate synthase in the stool of the subject. In a further aspect, the method includes measuring less than 1000 copies per million of 2-C-methyl-D-erythritol 2,4-cyclodiphosphate synthase in the stool of the subject. In an additional aspect, the method includes measuring less than 400 copies per million of L-rhamnose isomerase in the stool of the subject. In an alternate aspect, the method includes measuring less than 360 copies per million of L-rhamnose isomerase in the stool of the subject.

The method includes measuring a level of one or more amino acids in stool or liver tissue from the subject. In some aspects, the method includes measuring depletion of cysteine, homocysteine, S-adenosylhomocysteine, tryptophan, or a combination thereof in stool of the subject. In other aspects, the method includes measuring depletion of homocysteine, S-adenosylhomocysteine, kynurenine, or a combination thereof in liver tissue of the subject. In additional aspects, the method includes measuring an increase in serine, asparagine, or a combination thereof in stool of the subject. For example, the method can include measuring greater than about 0.05% relative abundance of serine among amino acids in stool of the subject, measuring less than about 0.005% relative abundance of cysteine among amino acids in stool of the subject, measuring a ratio of cysteine to serine of less than about 0.2 in stool of the subject, measuring a less than 0.005% relative abundance among amino acids of homocysteine in liver tissue of the subject, measuring less than 0.005% relative abundance among amino acids of homocysteine in stool of the subject, measuring less than 0.05% relative abundance among amino acids of S-adenosylhomocysteine in stool of the subject, measuring less than 4% relative abundance among amino acids of taurine in stool of the subject, measuring less than 0.5% relative abundance among amino acids of S-adenosylhomocysteine in liver tissue of the subject, measuring greater than 0.1% relative abundance among amino acids of asparagine in stool of the subject, measuring less than 3% relative abundance among amino acids of tryptophan in stool of the subject, measuring greater than 0.15% relative abundance among amino acids of kynurenine in liver tissue of the subject, measuring a kynurenine to tryptophan ratio of at least 0.05 in liver tissue of the subject, or a combination thereof.

After the subject has been identified as having or being at risk of developing steatosis, nonalcoholic steatohepatitis (NASH), nonalcoholic fatty liver disease (NAFLD), liver fibrosis, or cirrhosis, the subject is then optionally be administered a composition of the present disclosure, such as a composition with one or more hepatoprotective bacteria disclosed herein.

In some aspects, the method includes administering a diet and/or a dietary supplement that supports the growth or maintenance of a healthy gut microbiome in the subject. Examples of such diets include prebiotics such as certain fructans, galactans, and trisaccharides, magnesium, fish oil, L-glutamine, and vitamin D, as well as similar nutrients which promote gut microbiome health. In some aspects, the method includes administering a dietary supplement that supports the growth or maintenance of Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, or a combination of species thereof, for example by providing vitamin K, heme, flavins, lactic acid, or pyruvate.

In some aspects, the method includes administering an antimicrobial such as an antibiotic, an antifungal, or an antiviral to kill or slow the growth of a microbe or microbes whose amounts are desired to be decreased in the gut of the subject. In some aspects, the antimicrobial kills or slows the growth of a bacterium in the subject and does not kill or slow the growth of bacteria of the composition. In some aspects, the antimicrobial is an antifungal agent, such as an agent which is suitable for oral administration and which kills Candida albicans, a microbe prevalent in many human digestive tracts.

As used herein, the terms “a,” “an,” and “the” include plural reference unless the context dictates otherwise.

As used herein and in the claims, the terms “comprising,” “containing,” and “including” are inclusive, open-ended and do not exclude additional unrecited elements, compositional components or method steps. Accordingly, the terms “comprising” and “including” can be taken to encompass the comparably more restrictive terms “consisting of” and “consisting essentially of.”

As used herein, the terms “about” and “approximately,” in reference to a number, denote ranges encompassing ±10%, ±5%, or ±1% about the number unless otherwise stated or otherwise evident from the context (e.g., where such number would exceed 100% of a possible value).

As used herein, the term percent “identity,” in the context of two or more nucleic acid sequences, can refer to two or more sequences or subsequences that have a specified percentage of identical nucleotides when compared following alignment and optionally when further fit with gaps in one or more of the sequences to achieve maximum correspondence. As nonlimiting examples, identity between two or more nucleic acid sequences may be determined using GCG (Devereux, J., et al., Nucleic Acids Research, 12(1):387 (1984)), BLASTN and FASTA (Altschul, S. F. et al., J. Molec. Biol., 215:403410 (1990). Percent “identity” can pertain to a region of a sequence (e.g., a gene within a chromosome), or can apply to the full length of a sequence. In some aspects, a portion of a first sequence is compared against the entirety of a second sequence.

As used herein, the term “subject” can denote an intended recipient of a composition, treatment, or method. A subject can be an animals such as a mammal. Suitable subjects include, but are not limited to, primates (e.g., humans), cows, horses, dogs, cats, rabbits, rats, mice and the like. In some embodiments, the subject is a human.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the terms “treat”, “treating” and “treatment” can denote methods of alleviating or abrogating a disease or associated symptoms of a disease.

As used herein, “pharmaceutically acceptable” denotes that the carrier, diluent or excipient are compatible with the other ingredients of the formulation and not deleterious to the recipient thereof. For example, a pharmaceutically acceptable carrier of a bacterium intended for live administration may be an ingredient which does not kill or mutate the bacterium, and which is not deleterious to the intended recipient.

A composition can be administered alone or in combination with other treatments, either simultaneously or sequentially dependent upon the condition to be treated.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

### EXAMPLES

The following examples are provided to further illustrate the embodiments of the present invention but are not intended to limit the scope of the invention. While they are typical of those that might be used, other procedures, methodologies, or techniques known to those skilled in the art may alternatively be used.

### Example 1

### Gut Microbiome Profiling in a Human Population Disproportionately Affected By NASH

This example illustrates gut microbiome profiling in a human population with a high prevalence of NASH. Shotgun metagenomic sequencing was performed on stool samples from 340 randomly selected subjects from the CCHC. Among them, 48 (14.1%) had liver fibrosis (LSM ≥7.1 kPa), and 29 (8.5%) had advanced liver fibrosis (LSM ≥8.8 kPa), measured by VCTE-Fibroscan. To identify bacteria enriched or depleted in subjects with liver fibrosis or advanced liver fibrosis, effect size (LEfSe analysis) was performed from phylum to species level.

### Methods

The study included 340 participants from the CCHC (Fisher-Hoch et al. Prev Chronic Dis, 2010; 7: A532010), a population-based cohort disproportionally affected by NAFLD/NASH and liver fibrosis. Shotgun metagenomic sequencing was performed on stool samples from all subjects. Among them, 102 (30.0%) subjects were male, 187 (55.2%) were obese and 124 (37.3%) were diabetic. For each subject, VCTE (FibroScan®) was used for detection of steatosis and liver fibrosis. As summarized in TABLE 1, a total of 229 subjects (67.6%) had liver steatosis and 48 subjects (14.1%) had liver fibrosis. In this table, data are presented as frequency (%) for categorical variables, or mean (range)—median for continuous variables.

TABLE 1

| Parameters | Values | |
|---|---|---|
| Male (n = 340) | 102 | (30.0%) |
| Age (n = 340) | 55.1 | (18.0-89.0) − 57.0 |
| BMI (n = 339) | 31.4 | (16.7-50.0) − 30.8 |
| Obese (n = 339) | 187 | (55.2%) |
| Diabetes (n = 332) | 124 | (37.3%) |
| HbA1c (%) (n = 338) | 6.6 | (4.8-16.0) − 6.0 |
| Waist circumference (cm) (n = 340) | 104.1 | (71.0-143.0) − 104.0 |
| Waist-to-hip ratio (n = 322) | 0.9 | (0.7-1.1) − 0.9 |
| Hypertension (n = 340) | 118 | (34.7%) |
| FibroScan CAP (dB/m) (n = 339) | 290.7 | (100.0-400.0) − 298.0 |
| Liver steatosis (CAP > 268) (n = 339) | 229 | (67.6%) |
| FibroScan LSM (kPa) (n = 340) | 5.7 | (1.9-45.5) − 4.6 |
| Liver fibrosis (LSM ≥ 7.1 kPa) | 48 | (14.1%) |
| Advanced fibrosis (LSM > 8.8 kPa) | 29 | (8.5%) |
| (n = 340) | | |
| NAFLD score (n = 331) | −1.2 | (−5.5-8.9) − −1.1 |
| APRI (n = 337) | 0.3 | (0.1-2.3) − 0.2 |
| Alcohol intake (g/day) (n = 323) | 3.4 | (0.0-325.0) − 0.0 |

Drinking status (n = 323)

| | | |
|---|---|---|
| Never | 213 | (65.9%) |
| Moderate | 94 | (29.1%) |
| Heavy | 16 | (5.0%) |

Smoking status (n = 323)

| | | |
|---|---|---|
| Never | 233 | (72.1%) |
| Former | 67 | (20.7%) |
| Current | 23 | (7.1%) |

Blood tests

| | | |
|---|---|---|
| AST (U/L) (n = 337) | 21.7 | (6.0-205.0) − 19.0 |
| Abnormal AST (n = 337) | 29 | (8.6%) |
| ALT (U/L) (n = 337) | 32.4 | (12.0-173.0) − 27.0 |
| Abnormal ALT (n = 337) | 114 | (33.8%) |
| Total bilirubin (mg/dL) (n = 338) | 0.5 | (0.1-1.9) − 0.5 |
| Creatinine (mg/dL) (n = 338) | 0.8 | (0.4-5.0) − 0.7 |
| Albumin (mg/dL) (n = 338) | 3.9 | (3.0-4.6) − 3.9 |
| Alkaline phosphatase (U/L) (n = 338) | 90.1 | (38.0-165.0) − 86.0 |
| Fasting glucose (mg/dL) (n = 323) | 114.0 | (70.0-360.0) − 97.0 |
| Triglycerides (mg/dL) (n = 338) | 153.0 | (33.0-1596.0) − 126.0 |
| Total cholesterol (mg/dL) (n = 337) | 184.8 | (50.0-318.0) − 185.0 |
| HDL cholesterol (mg/dL) (n = 338) | 51.6 | (0.0-109.0) − 49.5 |
| LDL cholesterol (mg/dL) (n = 332) | 104.0 | (8.0-204.0) − 105.0 |
| Platelets (×10⁹/L) (n = 338) | 251.2 | (116.0-480.0) − 247.0 |

Subjects positive for hepatitis B or C virus or who had antibiotic, probiotic or proton pump inhibitor use within 30 days of stool collection, were excluded. Written informed consent was obtained from each participant and the study protocol was approved by the Committees for the Protection of Human Subjects, at participating institutions. The subjects were assessed for steatosis with vibration-controlled transient elastography (VCTE) (FibroScan® 502 Touch or FibroScan® 530 Compact, Echosens), and for liver fibrosis with liver stiffness measurements (LSM, kiloPascals, kPa). Significant liver fibrosis (F2-F4) was defined as LSM ≥7.1 kPa, while advanced fibrosis (F3-F4) was defined as LSM ≥8.8 kPa, as described in Wong et al. (Hepatology, 2010; 51: 454-62). LSM measurements were considered inconclusive if <10 valid measures or interquartile range-to-median ratio >0.3. Stool samples were collected from all participants using the OMNIgene® GUT stool collection kit (DNA Genotek, Ontario, Canada) and stored frozen at −80° C. until sequenced.

### Statistical Analysis

To identify bacteria with altered abundance in CCHC subjects with liver fibrosis or advanced liver fibrosis, differences in bacterial abundance between comparison groups were assessed using the linear discriminant analysis (LDA) effect size (LefSe) tool (Segata et al. Genome Biol, 2011; 12: R60), with p<0.05 and log 10 LDA score >2 considered significant. Taxa were included in analysis if they had >0.1%. abundance in at least 25% of samples in either comparison group. The remaining statistical analyses were performed in R (version 4.1.2; R Foundation for Statistical Computing, Vienna, Austria).

Additional differential abundance analysis of taxa was performed with ANCOM v2.1 (Kaul et al. Frontiers in Microbiology, 2017; 8:2114), where an FDR significance threshold of 0.2 was used for calculation of W statistics. W statistics greater than or equal to the 60^{th} percentile of the W distribution were considered significant. To determine associations between individual species and liver fibrosis or advanced liver fibrosis, logistic regression was performed. The “glm.fit” function was used to obtain odds ratios (ORs) adjusted for age and gender (AOR) and 95% confidence intervals (CIs). For Bacteroides caccae, Bacteroides ovatus, Bacteroides uniformis, Bacteroides finegoldii, Alistipes onderdonkii and Anaerobutyricum hallii, the AORs for liver fibrosis/advanced liver fibrosis were calculated for subjects in which each species was present or absent. For Parabacteroides distasonis and Gemmiger formicilis, AORs for liver fibrosis and advanced liver fibrosis were calculated respectively for subjects with relative abundance in the highest or lowest tertile.

### Mice

Animal procedures were carried out in accordance with the policies and regulations of the Institutional Animal Care and Use Committee at the Baylor College of Medicine. Eight-week-old male, germ-free C57BL/6J mice were purchased from, and housed at, the Germ Free Facility of the Baylor College of Medicine Gnotobiotics Core, under standard conditions of temperature, humidity and light control. Mice were group-housed. One flexible film isolator was used per experimental group and all technical manipulations were conducted while maintaining GF and gnotobiotic conditions.

### Bacterial Cell Culture

To design a clinically relevant bacterial consortium, for each species selected for treatment in the mouse study, the strain that was the most abundant in the human cohort data was used if commercially available. Parabacteroides distasonis (ATCC 8503), Anaerobutyricum hallii (ATCC 27751), Gemmiger formicilis (ATCC 27749), Bacteroides caccae (ATCC 43185), Bacteroides ovatus (ATCC 8483), and Bacteroides uniformis (ATCC 8492) were purchased from the American Type Culture Collection (ATCC). Bacteroides finegoldii (DSM 17565) and Alistipes onderdonkii subsp. vulgaris (DSM 108977) were purchased from the Leibniz-Institute DSMZ-German Collection of Microorganisms and Cell Cultures. Each bacterial strain was grown as monocultures in a Whitley A45 anaerobic workstation (10% H2, 5% CO2 and 85% N2), on trypticase soy agar, Brucella agar, Columbia agar or BYEM10+mucin agar. Prior to each preparation of inoculum, the identity of each bacterial strain was confirmed by biotyping using a BRUKER Matrix-Assisted Laser Desorption/Ionization Time-Of-Flight (MALDI-TOF) mass spectrometry. Bacterial cells were resuspended in PBS, quantified using a Nexcelom Cellometer cell counter with Syto BC green and propidium iodide staining, and combined for a preparation of inoculum containing 5×10⁸ CFU of each strain/ml. An aliquot of each preparation was taken prior to inoculation and the bacterial pellet was stored at −80° C. until sequenced.

### Results

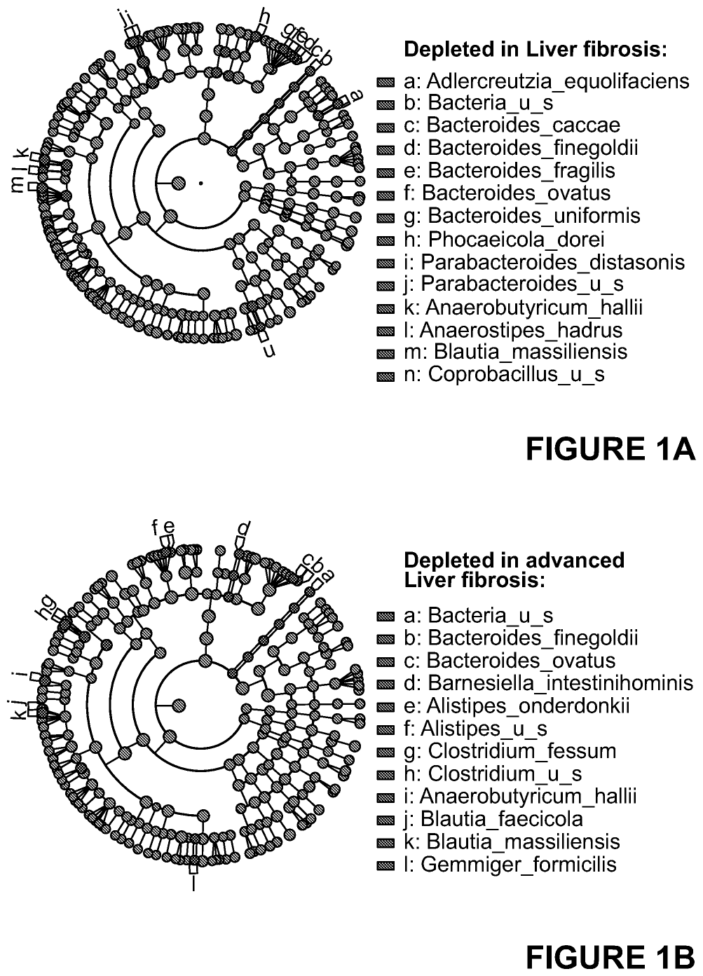

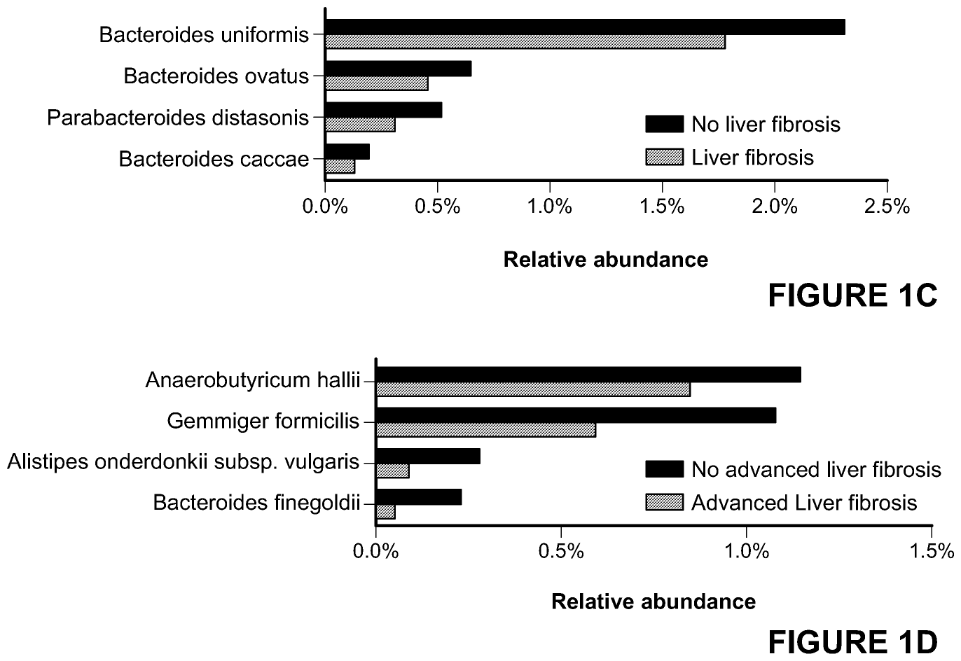

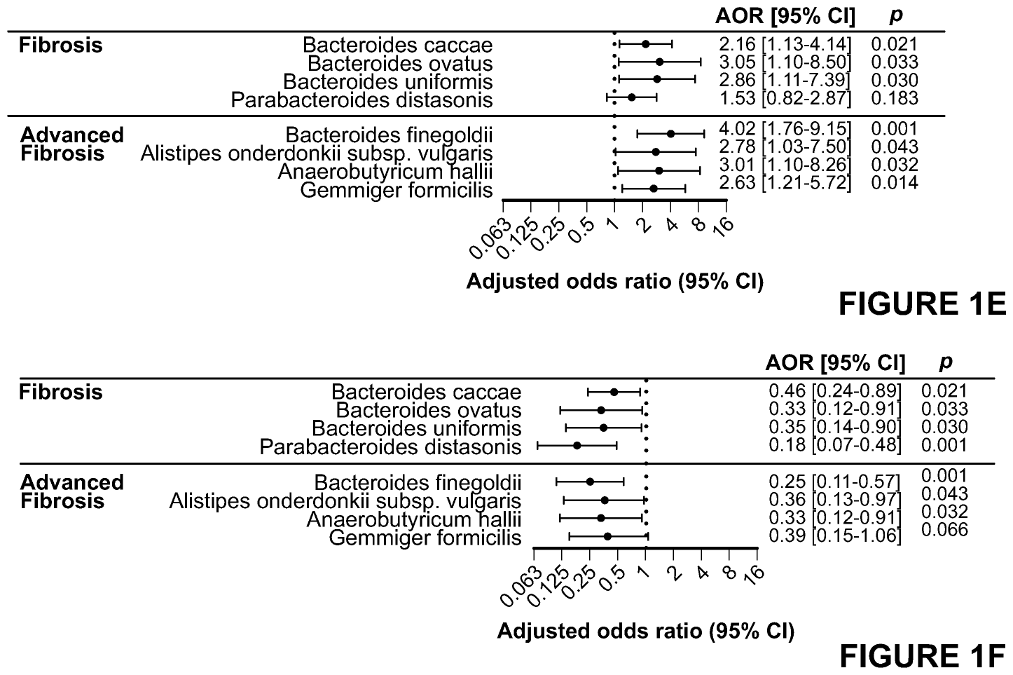

FIGS. 1A-1F display results of the microbiome profiling analyses. FIGS. 1A-1B are cladograms showing species significantly depleted in subjects with liver fibrosis (FIG. 1A) and subjects with advanced liver fibrosis (FIG. 1B) as assessed by the linear discriminant analysis (LDA) effect size (LEfSe) algorithm. FIGS. 1C-1D provide mean relative abundances of significant taxa in subjects with and without liver fibrosis (FIG. 1C), and subjects with and without advanced liver fibrosis (FIG. 1D). FIG. 1E provides a Forest plot which summarizes risk for liver fibrosis or advanced liver fibrosis as a function of Bacteroides caccae, Bacteroides ovatus, Bacteroides uniformis, Bacteroides finegoldii, Alistipes onderdonkii and Anaerobutyricum hallii absence and Parabacteroides distasonis and Gemmiger formicilis depletion. FIG. 1F provides a Forest plot summarizing the protective effects of Bacteroides caccae, Bacteroides ovatus, Bacteroides uniformis, Bacteroides finegoldii, Alistipes onderdonkii, Anaerobutyricum hallii, Parabacteroides distasonis, and Gemmiger formicilis against liver fibrosis or advanced liver fibrosis. In FIG. 1E-1F, adjusted odds ratios (AORs) and 95% confidence intervals (95% CI) are shown after adjusting for age and gender.

At the species level, 14 taxa were identified as depleted in subjects with liver fibrosis (FIG. 1A) and 12 taxa were identified as depleted in subjects with advanced liver fibrosis (FIG. 1B).

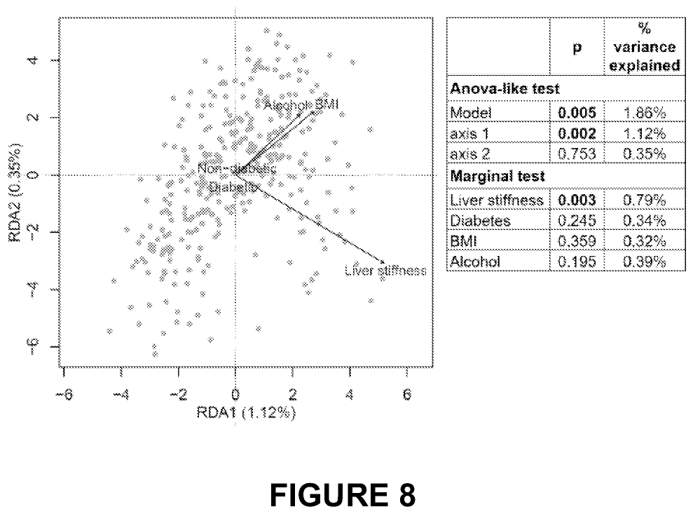

The contributions of clinical parameters to variation in the fibrosis-associated microbiome signature are shown in FIG. 8. To determine the confounding effect of common comorbidities on the liver-fibrosis associated microbiome signature, redundancy analysis was performed with FibroScan liver stiffness, diabetes status, BMI and alcohol intake as explanatory variables, and relative abundance of the 20 species associated with liver fibrosis and/or advanced liver fibrosis (in FIGS. 1A-1B) as response variables. ANOVA-like significance tests of the model, axes and explanatory variables were performed to determine which of these clinical variables contributed significantly to abundance variation of the 20 species across samples.

Overall, the model was significant (p=0.005). However, among the individual clinical variables, only liver stiffness was significant (p=0.003). Of these 20 species, 12 and 8 were considered significant by ANCOM analysis for liver fibrosis and advanced liver fibrosis, respectively (FDR<0.2). Bacterial species depleted in subjects with liver fibrosis included Bacteroides species, Parabacteroides distasonis, Anaerobutyricum hallii, Anaerostipes hadrus, Adlercreutzia equolifaciens and Blautia massiliensis, while bacterial species depleted in subjects with advanced liver fibrosis included Bacteroides finegoldii, Alistipes onderdonkii, Anaerobutyricum hallii and Gemmiger formicilis. The relative abundance and magnitude of change with liver fibrosis for Bacteroides caccae, Bacteroides ovatus, Bacteroides uniformis and Parabacteroides distasonis, or with advanced liver fibrosis for Anaerobutyricum hallii, Gemmiger formicilis, Alistipes onderdonkii subsp. Vulgaris and Bacteroides finegoldii are shown in FIGS. 1C-1D, respectively.

In addition, the strength of the associations between absence (Bacteroides caccae, Bacteroides ovatus, Bacteroides uniformis, Bacteroides finegoldii, Alistipes onderdonkii, Anaerobutyricum hallii) or abundance in the lowest tertile (Parabacteroides distasonis, Gemmiger formicilis) with liver fibrosis/advanced liver fibrosis were further determined by logistic regression analysis, adjusting for age and gender (FIG. 1E). The strongest abundance changes and associations of absence/low abundance with liver fibrosis/advanced liver fibrosis were observed for Bacteroides ovatus for liver fibrosis (fold change (FC)=−1.44; AOR=3.05 [95% CI=1.10-8.50], p=0.033), Bacteroides finegoldii for advanced liver fibrosis (FC=−4.23; AOR=4.02 [95% CI=1.76-9.15],p=0.001) and Anaerobutyricum hallii for advanced liver fibrosis (FC=−1.35; AOR=3.01 [95% CI=1.10-8.26],p=0.032). The presence of Bacteroides caccae, Bacteroides ovatus, Bacteroides uniformis, Bacteroides finegoldii, Alistipes onderdonkii, Anaerobutyricum hallii and abundance within the highest tertile of Parabacteroides distasonis, Gemmiger formicilis was associated with reduced risk of liver fibrosis/advanced liver fibrosis (FIG. 1F). These eight bacterial species were therefore subsequently selected for in vivo studies.

### Example 2

Microbiome Changes Associated with Liver Fibrosis

This example illustrates NASH induction with a methionine- and choline-deficient (MCD) diet in germ-free mice. While the MCD diet has been used extensively to induce NASH in conventional and specific pathogen-free (SPF) mice, its ability to induce NASH under germ-free conditions had not previously been assessed. To determine the relevance of the MCD-induced NASH model to test anti-fibrotic bacteria, eight-week-old GF and SPF mice were fed normal chow (GF and SPF groups), or MCD (GF-MCD and SPF-MCD) diets for 6 weeks. 5 to 10 mice were included in each treatment group.

FIG. 2A summarizes histological measurements performed on GF mice under regular diet (leftmost in each plot), GF mice after 6 weeks of MCD (second from left in each plot), SPF mice under regular diet (second from right in each plot), and SPF mice after 6 weeks of MCD (rightmost in each plot), with the top-left plot summarizing fibrosis stage, the top-middle plot summarizing steatosis, the top-right plot summarizing inflammation, the bottom-left plot summarizing ballooning, the bottom-middle plot summarizing ceroid laden macrophage counts, and the bottom-right plot summarizing NAFLD activity scores. The livers showed a significant induction of liver fibrosis, liver steatosis, inflammation, ceroid laden macrophages and NAFLD Activity Score for both SPF-MCD and GF-MCD mice (liver fibrosis: p<0.001 for SPF-MCD and p=0.034 for GF-MCD; liver steatosis: p<0.001 for both SPF-MCD and GF-MCD; inflammation: p=0.005 for SPF-MCD and p<0.001 for GF-MCD; ceroid laden macrophages: p<0.001 for SPF-MCD and p=0.002 for GF-MCD; NAFLD Activity Score: p<0.001 for both SPF-MCD and GF-MCD).

Ballooning hepatocyte degeneration (“ballooning”) was also measured in GF and SPF mice. In contrast to liver fibrosis and steatosis, ballooning scores reached significance in GF mice (p=0.038) but were not significantly induced by MCD in SPF mice but. While liver steatosis, ballooning and NAFLD Activity Scores were not significantly different between SPF-MCD and GF-MCD mice, the induction of liver fibrosis, inflammation and ceroid laden macrophages by MCD were significantly lower in GF-MCD mice compared to SPF-MCD mice (liver fibrosis: p=0.008; inflammation: p=0.037; ceroid laden macrophages: p=0.022).

FIG. 2B summarizes liver necropsy measurements of GF mice under regular diet (leftmost in each plot), GF mice after 6 weeks of MCD (second from left in each plot), SPF mice under regular diet (second from right in each plot), and SPF mice after 6 weeks of MCD (rightmost in each plot), with the top left plot providing liver-to-bodyweight ratios (%), the top right plot providing colon lengths (cm), the bottom left plot providing cecum weights (g), and the bottom right plot providing cecum to body weight ratios (%). While the liver to body weight ratio was significantly decreased in SPF-MCD compared to SPF mice (FC=−1.18, p=0.017), the ratio increased in GF-MCD compared to GF mice (FC=1.13, p=0.002). In contrast, colon length, cecum weight and the cecum to body weight ratio were all significantly decreased in both SPF and GF mice following MCD treatment. Colon length was reduced by FC=−1.16 and FC=−1.39 (p<0.001) in MCD-treated SPF and GF mice, respectively; cecum weight was reduced by FC=−2.92 and FC=−2.37 (p<0.001) while cecum to body weight ratio was reduced by FC=−1.76 and FC=−1.42 (p<0.001). Overall, these results suggest that MCD induced liver fibrosis and NASH in GF mice despite a lack of gut microbiome. However, MCD induced liver fibrosis, macrophages infiltration and inflammation in GF mice to a significantly lower degree than in SPF mice, suggesting a contribution of the gut microbiome to these specific histological parameters.

### Example 3

### Bacterial Inoculation Against Liver Fibrosis in NASH-Afflicted Mice

This example extends the shotgun metagenomic sequencing analysis from Example 1 to in vivo analysis of liver fibrosis in NASH-afflicted mice. Bacteria with the largest effect sizes for reducing liver fibrosis from the previous examples were selected for in vivo analyses in mice. For these analyses, Bacteroides caccae, Bacteroides ovatus, Bacteroides uniformis, Bacteroides finegoldii, Alistipes onderdonkii, Anaerobutyricum hallii, Parabacteroides distasonis, and Gemmiger formicilis were administered to eight-week old mice prior to NASH-induction by MCD diets.

Bacterial Inoculation of Germ-Free Mice with Diet-Induced NASH

To design a clinically relevant bacterial consortium, the strains selected for treatment in the mouse study were the strains that were most abundant in the human cohort data, if commercially available. A consortium consisting of the eight selected bacterial strains (one for each species: Bacteroides caccae, Bacteroides ovatus, Bacteroides uniformis, Bacteroides finegoldii, Alistipes onderdonkii, Parabacteroides distasonis, Anaerobutyricum hallii, and Gemmiger formicilis) was administered by oral gavage to eight-week old male, GF mice (GF-MCD-B).

Eight-week-old male, germ-free C57BL/6J mice, were housed at the Baylor College of Medicine Gnotobiotics Core. One flexible film isolator was used per experimental group and all technical manipulations were conducted by facility staff to maintain germ-free and gnotobiotic conditions. Specific pathogen-free (SPF) C57BL/6J mice were also acquired and housed at the Baylor College of Medicine Gnotobiotics Core.

As consecutive inoculations of bacteria may improve colonization in the gut of germ-free (GF) mice, mice were inoculated with the bacterial consortium three times in 5-day intervals. On days 0, 5 and 10, mice in the germ-free control group (GF+MCD) (n=11) were administered vehicle control of 200 μl PBS by oral gavage, while mice in the treated group (GF+MCD+bact) (n=10) were inoculated by oral gavage with a pool containing 1×10⁸ CFU of each of the 8 bacterial strains, in a total volume of 200 μl PBS. Ten to eleven mice were included in each group. On day 14, mice were switched from regular chow (autoclaved Select Rodent Diet 50 IF/6F, #5VOF, LabDiet) to the methionine- and choline-deficient (MCD) diet (#A02082002BR from Research Diets, double irradiated, with 1.5× vitamins) for the induction of NASH and liver fibrosis (Wang et al. Front Physiol, 2021; 12: 687744; Fisher et al. Gastroenterology, 2014; 147: 1073-83 e6; Im et al. Hepatology, 2021; 74: 1884-901). Stool was collected and snap-frozen on days 0 and 28, then stored at −80° C. until sequenced. Body weight was monitored twice weekly. Mice were sacrificed 8 weeks after first inoculation. At time of sacrifice, stool, serum, liver tissue, intestinal tissue and intestinal contents were collected. For additional control groups, age-matched germ-free mice on normal diet (GF, n=7), SPF mice on normal diet (SPF, n=5) and SPF mice on 6 weeks of MCD (SPF+MCD, n=10) were also sacrificed, and the same tissues were collected. Snap-frozen stool samples were subjected to shotgun metagenomic sequencing (CosmosID, Inc). Formalin-fixed paraffin-embedded (FFPE) liver sections were sectioned and stained with H&E and Masson's trichome, and histology was blindly assessed and scored by a collaborating pathologist for NAFLD score components (steatosis, ballooning, inflammation), ceroid laden macrophages and fibrosis. The NAFLD Activity Score (NAS) was calculated as an unweighted sum of steatosis (0-3), ballooning (0-2), and lobular inflammation (0-3) scores.

### Stool DNA Extraction, Shotgun Metagenomics Sequencing and Bioinformatic Analysis

Shotgun metagenomic sequencing of all human stool samples, mouse stool pellets and bacterial inocula were subjected to shotgun metagenomic sequencing (CosmosID Inc., Rockville, Maryland). DNA was isolated using the QIAGEN Dneasy PowerSoil Pro Kit (Qiagen) and quantified by Qubit (ThermoFisher). DNA libraries were prepared using the Illumina Nextera XT library preparation kit. Libraries were assessed with Qubit (ThermoFisher) and sequenced on an Illumina HiSeq platform using 150 bp paired-end sequencing, to a sequencing depth of 12 million reads (±20%). Unassembled sequencing reads were directly analyzed by the CosmosID bioinformatics platform, which utilizes a high performance data-mining K-mer-based algorithm and curated genomic databases as described elsewhere (Ponnusamy et al. Proc Natl Acad Sci USA, 2016; 113: 722-7), to perform taxonomic profiling at strain-level resolutions. Bacterial abundance scores and relative abundances were used for downstream analysis. Statistical differences in histology scores and necropsy measurements between groups were assessed by the Mann-Whitney U test for ordinal histology scores, and the unpaired t-test for continuous variables. Statistical differences in amino acids were assessed by the Mann-Whitney U test. For all tests, p<0.05 was considered significant.

For functional annotation of the metagenome, initial quality control, adapter trimming and preprocessing of metagenomic sequencing reads were performed using Bbduk (https://jgi.doe.gov/data-and-tools/bbtools/). Quality-controlled reads were subjected to a translated search against the protein sequence database UniRef 90. The mapping of metagenomic reads to gene sequences were weighted by mapping quality, coverage and gene sequence length to estimate community wide weighted gene family abundances. Gene families were annotated to MetaCyc⁷¹ reactions (Metabolic Enzymes) to reconstruct and quantify MetaCyc metabolic pathways.⁷¹ Abundance values were normalized using Total-sum scaling normalization to produce “Copies per million”. Pathways and enzymes with CPM<10 across all inocula and stool samples were excluded from downstream analyses.

Mouse Liver DNA Extraction, 16S rRNA Amplicon Sequencing and Bioinformatic Analysis

Mouse liver samples were analyzed by 16S sequencing at the MD Anderson Cancer Center Microbiome Core Facility. Microbial genomic DNA was extracted using the DNeasy Powersoil Pro DNA kit (Cat No. 47014, QIAGEN). Liver tissue (100-200 mg) was homogenized using mechanical and chemical methods. The lysed cells were treated with Inhibitor Removal Technology solution. The genomic DNA was then captured on a silica membrane in a spin-column format, followed by washing and elution steps. Methods from the Earth Microbiome Project were adapted to generate 16S v4 amplicon libraries. PCR amplification of microbial DNA was carried out using 515F and 806R primer constructs containing sequencing-ready barcodes and adapter sequences. The quality and quantity of the barcoded amplicons were assessed using an Agilent 4200 TapeStation system (Agilent) and Qubit Fluorometer (Thermo Fisher Scientific). The amplicons were pooled in equimolar ratios. The pooled libraries were quantified using a Qubit fluorometer, and their molarity was calculated based on the amplicon size. Sequencing was performed on the MiSeq platform (Illumina) using the 2×250 bp paired-end protocol, resulting in paired-end reads with near-complete overlap. The custom sequencing primers used were: Read1: 5′-TATGGTAATTGTGTGYCAGCMGCCGCGGTAA-3′; Read2: 5′-AGTCAGCCAGCCGGA CTACNVGGGTWTCTAAT-3′; and index sequencing primer: AATGATACGGCGACC ACCGAGATCTACACGCT. Paired-end reads were de-multiplexed using QIIME. The paired-end reads were merged, followed by dereplication and length filtering using VSEARCH v2.17.1. De-noising and chimera calling were performed using the unoise3 command. Bacterial taxonomies were assigned using the SILVA database version 138. Library preparation failed for two liver samples from the GF-MCD group and two liver samples from the GF-MCD-B group.

### Whole Genome Annotations of Inoculated Bacterial Species

Raw genome sequencing files were downloaded from the ATCC Genome Portal for Parabacteroides distasonis ATCC 8503, Bacteroides caccae ATCC 43185, and Bacteroides ovatus ATCC 8483, or from the NCBI data hub for Bacteroides uniformis ATCC 8492 (GCF_900107315.1), Bacteroides finegoldii DSM 17565 (GCF_000156195.1), and Alistipes onderdonkii subsp. vulgaris DSM 108977 (GCF_006542645.1). Each raw sequencing file was annotated using the prokka pipeline [v1.14.16] available on the Proksee online tool.

### Amino Acids Profiling

Stool, cecum and liver extracts were prepared and analyzed by ultra-high-resolution mass spectrometry at the MD Anderson Metabolomics Core Facility. Samples (20-30 mg) were pulverized in liquid nitrogen, then homogenized with Precellys Tissue Homogenizer. A mixture of 17 stable isotope-labeled amino acids were spiked into each sample as an internal standard. Amino acids were extracted using 1 mL ice-cold 90/10 (v/v) acetonitrile/water with 0.1% formic acid. Extracts were centrifuged at 17,000 g for 5 min at 4° C., and supernatants were transferred to clean tubes, followed by evaporation to dryness under nitrogen. Dried extracts were reconstituted in 90/10 acetonitrile/water containing 1% formic acid, then 10 L was injected for analysis by liquid chromatography (LC)-MS. LC mobile phase A (MPA; weak) was acetonitrile containing 1% formic acid, and mobile phase B (MPB; strong) was water containing 50 mM ammonium formate. A Thermo Vanquish LC system included an Imtakt Intrada Amino Acid column (3 μm particle size, 150×2.1 mm) with column compartment kept at 30° C. The autosampler tray was chilled to 4° C. The mobile phase flow rate was 300 μL/min, and the gradient elution program was: 0-5 min, 15% MPB; 5-20 min, 15-30% MPB; 20-30 min, 30-95% MPB; 30-40 min, 95% MPB; 40-41 min, 95-15% MPB; 41-50 min, 15% MPB. The total run time was 50 min. Data were acquired using a Thermo Orbitrap Exploris 240 Mass Spectrometer under ESI positive ionization mode at a resolution of 240,000. Raw data files were imported to Thermo Trace Finder software for final analysis. Peaks with a signal-to-noise ratio <3 were considered undetected. The relative abundance of 37 amino acids was calculated by the individual peak area divided by total peak area.

### Results

### Inoculum

FIG. 3A summarizes the proportion of the 8 bacterial strains in the inoculums prepared on days 0 (left), 5 (middle), and 10 (right) days. The profiling confirmed a lack of contamination from other strains, and determined mean relative abundances of 22.9% for Bacteroides caccae, 13.7% for Bacteroides ovatus, 5.1% for Bacteroides uniformis, 15.3% for Bacteroides finegoldii, 10.6% for Alistipes onderdonkii, 15.7% for Parabacteroides distasonis, 2.9% for Anaerobutyricum hallii, and 8.1% for Gemmiger formicilis within the inoculums.

### Composition of Bacterial Pools Colonized in GF Mice

Following administration of the 8 bacterial strains and two weeks for colonization followed by NASH-induction with MCD-diets (FIG. 3C), shotgun metagenomic sequencing was performed on stool of the GF-MCD mice inoculated with the 8 bacterial strains. FIG. 3B provides relative abundances of Bacteroides caccae (top left plot), Bacteroides ovatus (top plot second from left), Bacteroides uniformis (top plot second from right), Bacteroides finegoldii (top right plot), Alistipes onderdonkii (bottom left plot), Parabacteroides distasonis (bottom plot second from left), Anaerobutyricum hallii (bottom plot second from right), and Gemmiger formicilis (bottom right plot) in GF mouse stool 0 (leftmost column in each plot), 14 (middle column in each plot), and 28 (rightmost column in each plot) days prior to first inoculation. These analyses confirmed a lack of gut microbiota in all mice on day 0, immediately prior to the first inoculation. At 28 days after first gavage, a lack of gut microbiota was again confirmed in all GF-MCD mice. On days 14 and 28, 6 of the 8 bacterial species successfully colonized the gut of the mice in the treated GF-MCD-bact group (Bacteroides finegoldii, Bacteroides caccae, Bacteroides ovatus, Bacteroides uniformis, Parabacteroides distasonis, Alistipes onderdonkii). These 6 strains were detected in all GF-MCD-bact mice at both 14 and 28 days after first inoculation, except for Paracteroides distasonis, which was detected in 8 out of 10 mice 28 days after first inoculation. At 14 days, Bacteroides ovatus was the most abundant strain (median relative abundance of 31.2%), followed by Bacteroides caccae (23.3%) and Bacteroides uniformis (15.0%). At 28 days, these remained the most abundant strains, with Bacteroides caccae becoming the most abundant strain (77.3%), followed by Bacteroides ovatus (11.2%) and Bacteroides uniformis (3.7%).

The shotgun metagenomic sequencing confirmed the presence of all eight bacterial strains in the inocula prepared on days 0, 5 and 10, as well as a lack of contamination from other strains (FIG. 3B). However, relative abundances of the eight bacterial species differed with an overall high abundance of Bacteroides caccae (mean=22.9%, range=12.9-32.5%), Parabacteroides distasonis (mean=15.7% range=9.2-28.8%) and Bacteroides finegoldii (mean=15.3%, range=10.3-21.7%), while abundance was low for Anaerobutyricum hallii (mean=2.9%, range=2.3-4.3%) and Gemmiger formicilis (mean=8.1%, range=2.0-18.5%).

It was also determined whether the inoculated bacteria could be detected in the liver of the treated mice. None of the eight inoculated bacteria were detected in the livers of GF-MCD mice. In contrast, all six species that successfully colonized the gut were detected in liver samples of GF-MCD-B mice. Similar to the gut microbiome at 28 days, Bacteroides caccae was the most abundant species in the liver, with presence detected in all eight GF-MCD-B mice and a median relative abundance of 86.5% (FIG. 3D), followed by Bacteroides uniformis (detected in the livers of six GF-MCD-B mice, median abundance of 2.2%) and Bacteroides ovatus (detected in the livers of five GF-MCD-B mice, median abundance of 3.7%). In contrast, Alistipes onderdonkii, Bacteroides finegoldii and Parabacteroides distasonis were detected in only three, two and one liver sample, respectively. Correlation analysis between relative abundances in the liver and in stool collected at day 28, showed that overall the most abundant species in stool were also the most abundant species in the liver (rₛ=0.75, p<0.001) (FIG. 3E).

Prevention of Liver Fibrosis by Selected Bacterial Species in Mice with MCD-Induced NASH

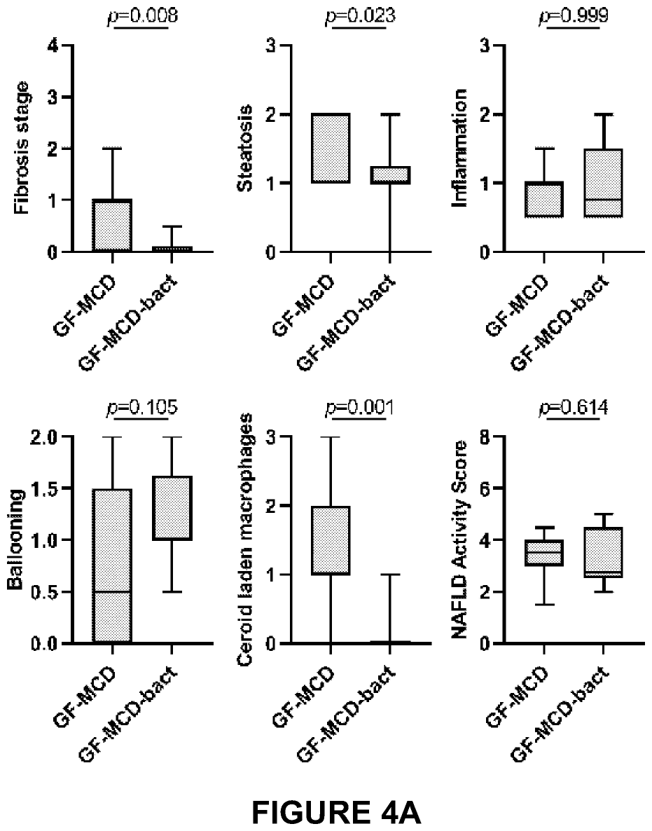

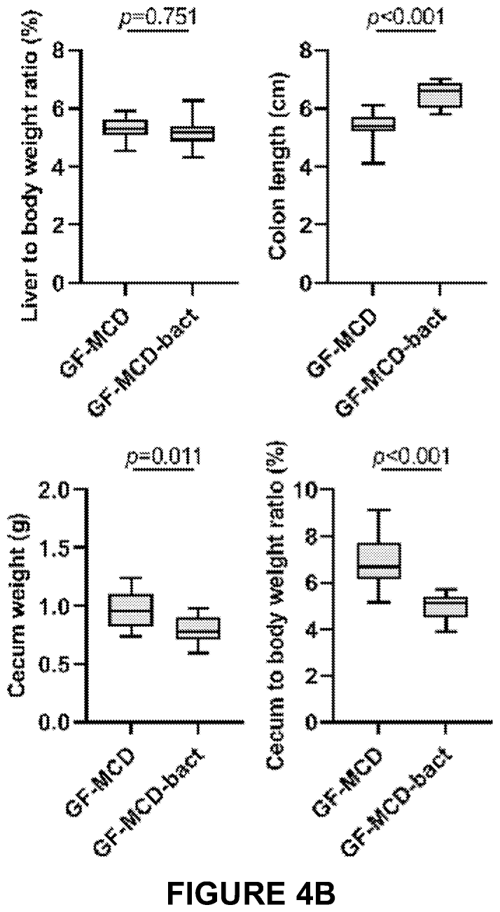

Diet was changed to MCD for both bacteria-treated GF mice (GF-MCD-bact) and control GF mice (GF-MCD) 4 days after the last inoculation, corresponding to 14 days after first inoculation. Mice were necropsied after 42 days of MCD. The results of the histological and necropsy measurements are summarized in FIG. 4A and FIG. 4B, respectively. In FIG. 4A, the top-left plot summarizes fibrosis stage, the top-middle plot summarizes steatosis, the top-right plot summarizes inflammation, the bottom-left plot summarizes ballooning, the bottom-middle plot summarizes ceroid laden macrophage counts, and the bottom-right plot summarizes NAFLD activity scores in bacteria- (left) and vehicle- (right) treated GF-MCD mice. In FIG. 4B, the top left plot summarizes liver-to-bodyweight ratios (%), the top right plot summarizes colon lengths (cm), the bottom left plot summarizes cecum weights (g), and the bottom right plot summarizes cecum to body weight ratios (%) in bacteria-(left) and vehicle- (right) treated GF-MCD mice.

Histological assessment of livers showed that while there was no significant change in inflammation, ballooning or NAFLD Activity Score, there was a significant reduction of liver fibrosis (median score of 0 in GF-MCD-bact mice vs 1 in GF-MCD mice, p=0.008), liver steatosis (1 versus 2, p=0.023) and ceroid laden macrophages (0 versus 1, p=0.001) in mice pre-treated by the bacterial pool (FIG. 4A). Notably, liver fibrosis was detected in only 2 out of the 10 (20%) GF-MCD-bact mice but in 8 out of 11 mice (73%) in the GF-MCD group had liver fibrosis. Additionally, while there was no significant change in the liver to body weight ratio, GF-MCD-bact mice had significantly increased colon length (6.6 vs 5.4 cm, p<0.001), decreased cecum weight (0.78 g vs 0.96 g, p=0.011) and decreased cecum to body weight ratio (5.1% vs 6.7%, p<0.001) compared to GF-MCD mice (FIG. 4B).

### Discussion

The studies disclosed herein utilized shotgun metagenomic sequencing of stool collected from a large set of subjects (n=340) selected from a population cohort disproportionally affected by NAFLD and liver fibrosis. To identify bacterial species with a potential to prevent liver fibrosis, these studies focused on bacterial species for which low abundance was significantly associated with increased risk of liver fibrosis and inversely, for which high abundance had a protective effect against liver fibrosis. Low abundance of Bacteroides species (Bacteroides caccae, Bacteroides finegoldii, Bacteroides ovatus, Bacteroides uniformis) and Parabacteroides distasonis were associated with increased risk of liver fibrosis. Additional discoveries included the association between low abundance of Alistipes onderdonkii subsp. Vulgaris, Anaerobutyricum hallii and Gemmiger formicilis with increased risk of liver fibrosis.

Subsequently, the abilities of eight bacterial strains selected from the human cohort data were tested for their abilities to prevent MCD-induced liver fibrosis in GF mice. The bacterial consortium consisted of Bacteroides caccae, Bacteroides ovatus, Bacteroides uniformis, Bacteroides finegoldii, Alistipes onderdonkii, Parabacteroides distasonis, Anaerobutyricum hallii, and Gemmiger formicilis. All eight strains were detected in the three inocula used to treat the mice, although with variable abundance. The variation in relative abundance across bacterial species and batches of inocula may be due to differential oxygen sensitivities between strains, or a difference in growth rates leading to changes in the composition of the inocula prior to processing and storage. Only six strains (Bacteroides caccae, Bacteroides ovatus, Bacteroides uniformis, Bacteroides finegoldii, Parabacteroides distasonis and Alistipes onderdonkii subsp. Vulgaris) successfully colonized the gut of these mice. Despite not being the most abundant in the inocula, Bacteroides ovatus displayed the highest relative abundance in stool at day 14. Conversely, Bacteroides caccae had the highest average abundance in the inocula, and was also the most abundant strain in stool at day 28. The difference in gut microbiome composition, 4 and 18 days after last inoculation, are in agreement with other studies in GF mice, where temporal variation in gut microbiome composition was reported in the first 1-2 weeks after inoculation. Lack of successful colonization by Anaerobutyricum halli and Gemmiger formicilis may be due to relatively low abundance in the inocula, loss of viability during preparation of the inocula, or lower competitiveness compared with the other bacterial strains in the consortium.

Interestingly, each of the six colonizing strains in the gut could also be detected in 12.5-100% of liver tissues after bacterial inoculation. Strains with higher abundance in the stool also had higher abundance in the liver. The major Bacteroidetes phylum in the gut was also detected as the most abundant phylum in the liver, and modulated hepatic immune cell infiltration and maturation. Pre-treatment of GF mice with the bacterial consortium significantly reduced liver steatosis, liver fibrosis, and the presence of ceroid laden macrophages in the liver. Ceroid accumulation in macrophages is considered a marker of liver injury and chronic oxidative stress. Introduction of the bacterial consortium also altered morphologic features of the gut, by way of increased colon length and decreased cecal weight. Shortened colon length is a marker of colonic inflammation, commonly reported in mouse models of colitis. The increased colon length upon bacterial inoculation suggested a possible improvement in colonic inflammatory status. However, histological assessment of the ileum and colon did not show significant differences in the histological scores between the control and treated mice (data not shown). A reduction in the total cecal weight also reflects an abrogation of the enlarged cecum that is a hallmark of GF mice.

### Example 4

Functional Mechanisms Correlating with Anti-Fibrotic Effect of the Inoculated Bacterial Consortium: a Human to Mouse Comparative Metagenomic Functional Analysis

To identify mechanisms by which the selected bacterial species may exert anti-fibrotic effects, comparative analysis was performed between the metagenomic functional profiles of human and mouse samples. Stool metagenomics functions negatively associated with liver fibrosis in the 340 cohort subjects were first characterized. Eight MetaCyc pathways and 26 MetaCyc enzymes were significantly depleted in subjects with liver fibrosis and advanced liver fibrosis. Their low abundance was significantly associated with increased risk for liver fibrosis or advanced liver fibrosis while their high abundance was significantly associated with protection against liver fibrosis or advanced liver fibrosis. MetaCyc pathways and enzymes associated with liver fibrosis or advanced liver fibrosis in the human cohort are summarized in TABLES 2 and 3, respectively. A total of 8 pathways and 26 enzymes displayed a significant negative association (by both Mann-Whitney and logistic regression) with both liver fibrosis and advanced fibrosis. Four pathways were found to be enriched in the stool of mice after bacterial inoculation, namely: pentose phosphate pathway (non-oxidative branch) (MetaCyc ID NONOXIPENT-PWY); CDP-diacylglycerol biosynthesis I (MetaCyc ID PWY-5667), preQ0 biosynthesis (MetaCyc IDPWY-6703), and CDP-diacylglycerol biosynthesis II (MetaCyc ID PWYO-1319). Furthermore, six enzymes were found to be enriched in the stool of mice after bacterial inoculation, namely: 2-oxoglutarate synthase (MetaCyc ID 1.2.7.3), cysteine synthase (MetaCyc ID 2.5.1.47), penicillin amidase (MetaCyc ID 3.5.1.11), N-acetylmuramic acid 6-phosphate etherase (MetaCyc ID 4.2.1.126), 2-C-methyl-D-erythritol 2,-4-cyclodiphosphate synthase (MetaCyc ID 4.6.1.12), and L-rhamnose isomerase (MetaCyc ID 5.3.1.14). Median abundances are shown in copies per million (CPM). Adjusted odds ratios (AORs) were adjusted for age and gender.

TABLE 2

| | | Liver fibrosis | | | | | | | |
|---|---|---|---|---|---|---|---|---|---|
| | | CPM | | | | | | | |
| | | No | CPM | | | AOR | | AOR | |
| MetaCyc ID | Description | fibrosis | Fibrosis | MW p | FC | (low) | P | (high) | P |

Pathways

| | | | | | | | | | |
|---|---|---|---|---|---|---|---|---|---|
| CITRULBIO- | L-citrulline biosynthesis | 403 | 173 | <0.001 | 0.43 | 4.33 | 0.001 | 0.23 | 0.001 |
| PWY | | | | | | (1.88-9.98) | | (0.10-0.53) | |
| NONOXIPENT- | pentose phosphate | 10178 | 9328 | 0.003 | 0.92 | 2.01 | 0.032 | 0.24 | 0.009 |
| PWY | pathway | | | | | (1.06-3.81) | | (0.08-0.70) | |
| | (non-oxidative branch) | | | | | | | | |
| PWY-1042 | glycolysis IV | 13372 | 12591 | 0.021 | 0.94 | 2.44 | 0.013 | 0.72 | 0.418 |
| | (plant cytosol) | | | | | (1.21-4.91) | | (0.33-1.59) | |
| PWY-4984 | urea cycle | 341 | 135 | <0.001 | 0.40 | 4.20 | 0.001 | 0.24 | 0.001 |
| | | | | | | (1.82-9.66) | | (0.10-0.55) | |
| PWY-5667 | CDP-diacylglycerol | 12390 | 11698 | 0.006 | 0.94 | 1.85 | 0.073 | 0.30 | 0.006 |
| | biosynthesis I | | | | | (0.95-3.61) | | (0.13-0.71) | |
| PWY-5695 | urate | 8411 | 7892 | 0.049 | 0.94 | 1.73 | 0.100 | 0.39 | 0.040 |
| | biosynthesis/inosine | | | | | (0.90-3.31) | | (0.16-0.96) | |
| | 5′- phosphate degradation | | | | | | | | |
| PWY-6703 | preQ0 biosynthesis | 5374 | 4686 | <0.001 | 0.87 | 3.25 | <0.001 | 0.31 | 0.007 |
| | | | | | | (1.68-6.29) | | (0.13-0.72) | |
| PWY0-1319 | CDP-diacylglycerol | 12390 | 11698 | 0.006 | 0.94 | 1.85 | 0.073 | 0.30 | 0.006 |
| | biosynthesis II | | | | | (0.95-3.61) | | (0.13-0.71) | |

Enzymes

| | | | | | | | | | |
|---|---|---|---|---|---|---|---|---|---|
| 1.1.1.18 | Inositol 2-dehydrogenase | 96 | 75 | 0.028 | 0.78 | 1.92 | 0.055 | 0.59 | 0.153 |
| | | | | | | (0.99-3.73) | | (0.28-1.22) | |
| 1.1.1.38, | Malate dehydrogenase | 1081 | 865 | 0.005 | 0.80 | 2.11 | 0.030 | 0.35 | 0.011 |
| 4.1.1.3 | (oxaloacetate- | | | | | (1.08-4.15) | | (0.16-0.79) | |
| | decarboxylating)l | | | | | | | | |
| | Oxaloacetate | | | | | | | | |
| | decarboxylase | | | | | | | | |
| 1.1.3.15 | (S)-2-hydroxy-acid | 105 | 77 | 0.007 | 0.73 | 1.88 | 0.052 | 0.39 | 0.021 |
| | oxidase | | | | | (1.00-3.55) | | (0.17-0.86) | |
| 1.14.13.39 | Nitric-oxide synthase | 122 | 102 | 0.011 | 0.84 | 1.90 | 0.067 | 0.44 | 0.036 |
| | (NADPH) | | | | | (0.96-3.78) | | (0.20-0.95) | |
| 1.17.7.3 | (E)-4-hydroxy- | 1341 | 1280 | 0.040 | 0.95 | 1.99 | 0.036 | 0.71 | 0.391 |
| | 3 -methylbut-2- | | | | | (1.05-3.78) | | (0.32-1.55) | |
| | enyl-diphosphate | | | | | | | | |
| | synthase (flavodoxin) | | | | | | | | |
| 1.2.7.1 | Pyruvate synthase | 94 | 75 | 0.047 | 0.80 | 1.52 | 0.211 | 0.59 | 0.199 |
| | | | | | | (0.79-2.92) | | (0.26-1.32) | |
| 1.2.7.3 | 2-oxoglutarate synthase | 98 | 69 | 0.008 | 0.71 | 2.37 | 0.011 | 0.30 | 0.015 |
| | | | | | | (1.22-4.60) | | (0.11-0.79) | |
| 1.2.7.8 | Indolepyruvate ferredoxin | 517 | 464 | 0.041 | 0.90 | 1.40 | 0.337 | 0.51 | 0.079 |
| | oxidoreductase | | | | | (0.70-2.80) | | (0.24-1.08) | |
| 1.2.99.2 | Carbon-monoxide | 356 | 287 | 0.007 | 0.81 | 2.40 | 0.007 | 0.59 | 0.155 |
| | dehydrogenase (acceptor) | | | | | (1.26-4.56) | | (0.29-1.22) | |
| 2.5.1.47 | Cysteine synthase | 1294 | 1216 | 0.003 | 0.94 | 1.85 | 0.073 | 0.25 | 0.011 |
| | | | | | | (0.94-3.64) | | (0.09-0.73) | |
| 2.7.1.11 | 6-phosphofructokinase | 2145 | 1955 | 0.002 | 0.91 | 2.41 | 0.009 | 0.51 | 0.079 |
| | | | | | | (1.24-4.68) | | (0.24-1.08) | |
| 2.7.2.8 | Acetylglutamate kinase | 1330 | 1267 | 0.045 | 0.95 | 1.79 | 0.077 | 0.64 | 0.210 |
| | | | | | | (0.94-3.40) | | (0.31-1.29) | |
| 2.7.7.41 | Phosphatidate | 1064 | 902 | 0.012 | 0.85 | 2.57 | 0.004 | 0.57 | 0.128 |
| | cytidylyltransferase | | | | | (1.36-4.86) | | (0.28-1.18) | |
| 3.2.1.21 | Beta-glucosidase | 1072 | 951 | 0.034 | 0.89 | 1.92 | 0.046 | 0.49 | 0.098 |
| | | | | | | (1.01-3.63) | | (0.21-1.14) | |
| 3.2.1.51 | Alpha-L-fucosidase | 124 | 85 | 0.011 | 0.69 | 2.20 | 0.016 | 0.51 | 0.080 |
| | | | | | | (1.15-4.17) | | (0.24-1.08) | |
| 3.2.1.55 | Non-reducing end alpha- | 213 | 163 | 0.014 | 0.77 | 1.87 | 0.056 | 0.49 | 0.063 |
| | L-arabinofuranosidase | | | | | (0.98-3.54) | | (0.23-1.04) | |
| 3.4.21.89 | Signal peptidase I | 2510 | 2313 | 0.005 | 0.92 | 2.26 | 0.017 | 0.31 | 0.006 |
| | | | | | | (1.16-4.42) | | (0.13-0.71) | |
| 3.5.1.11 | Penicillin amidase | 169 | 55 | <0.001 | 0.33 | 3.16 | <0.001 | 0.26 | 0.013 |
| | | | | | | (1.65-6.02) | | (0.09-0.76) | |
| 3.5.1.24 | Choloylglycine hydrolase | 283 | 224 | 0.002 | 0.79 | 2.45 | 0.008 | 0.38 | 0.019 |
| | | | | | | (1.27-4.73) | | (0.17-0.86) | |
| 3.5.4.12 | dCMP deaminase | 309 | 255 | 0.020 | 0.83 | 2.29 | 0.012 | 0.57 | 0.175 |
| | | | | | | (1.20-4.38) | | (0.25-1.28) | |
| 4.2.1.126 | N-acetylmuramic acid | 259 | 183 | 0.003 | 0.71 | 3.11 | 0.001 | 0.31 | 0.019 |
| | 6- phosphate etherase | | | | | (1.61-6.00) | | (0.12-0.83) | |
| 4.6.1.12 | 2-C-methyl-D-erythritol | 1173 | 988 | 0.007 | 0.84 | 2.26 | 0.013 | 0.26 | 0.003 |
| | 2,4-cyclodiphosphate | | | | | (1.19-4.29) | | (0.10-0.63) | |
| | synthase | | | | | | | | |
| 4.99.1.3 | Sirohydrochlorin | 92 | 74 | 0.021 | 0.80 | 1.81 | 0.089 | 0.42 | 0.029 |
| | cobaltochelatase | | | | | (0.91-3.58) | | (0.19-0.91) | |
| 5.3.1.14 | L-rhamnose isomerase | 431 | 344 | 0.032 | 0.80 | 1.99 | 0.036 | 0.40 | 0.022 |
| | | | | | | (1.05-3.79) | | (0.19-0.88) | |
| 6.3.5.1 | NAD(+) synthase | 195 | 169 | 0.019 | 0.87 | 1.91 | 0.049 | 0.52 | 0.091 |
| | (glutamine-hydrolyzing) | | | | | (1.00-3.65) | | (0.25-1.11) | |
| 6.3.5.11 | Cobyrinate a,c-diamide | 260 | 217 | 0.024 | 0.83 | 2.49 | 0.005 | 0.61 | 0.180 |
| | synthase (glutamine- | | | | | (1.31-4.73) | | (0.29-1.26) | |
| | hydrolyzing) | | | | | | | | |

TABLE 3

| | | Advanced liver fibrosis | | | | | | | |
|---|---|---|---|---|---|---|---|---|---|
| | | CPM No | CPM | | | | | | |
| | | advanced | Advanced | | | AOR | | AOR | |
| MetaCyc ID | Description | fibrosis | fibrosis | MW p | FC | (low) | P | (high) | P |

Pathways

| | | | | | | | | | |
|---|---|---|---|---|---|---|---|---|---|
| CITRULBIO- | L-citrulline biosynthesis | 375 | 247 | 0.010 | 0.66 | 2.44 | 0.027 | 0.36 | 0.045 |
| PWY | | | | | | (1.11-5.37) | | (0.13-0.98) | |
| NONOXIPENT- | pentose phosphate | 10115 | 8952 | 0.007 | 0.89 | 3.13 | 0.004 | 0.30 | 0.056 |
| PWY | pathway | | | | | (1.43-6.84) | | (0.09-1.03) | |
| | (non-oxidative branch) | | | | | | | | |
| PWY-1042 | glycolysis IV | 13349 | 11749 | 0.031 | 0.88 | 3.11 | 0.006 | 0.76 | 0.534 |
| | (plant cytosol) | | | | | (1.39-6.96) | | (0.33-1.79) | |
| PWY-4984 | urea cycle | 316 | 207 | 0.010 | 0.66 | 2.84 | 0.009 | 0.36 | 0.045 |
| | | | | | | (1.30-6.22) | | (0.13-0.98) | |
| PWY-5667 | CDP-diacylglycerol | 12403 | 11412 | 0.004 | 0.92 | 2.21 | 0.050 | 0.29 | 0.025 |
| | biosynthesis I | | | | | (1.00-4.89) | | (0.10-0.86) | |
| PWY-5695 | urate | 8404 | 7476 | 0.028 | 0.89 | 1.99 | 0.081 | 0.31 | 0.058 |
| | biosynthesis/inosine | | | | | (0.92-4.30) | | (0.09-1.04) | |
| | 5′- phosphate degradation | | | | | | | | |
| PWY-6703 | preQ0 biosynthesis | 5221 | 4764 | 0.019 | 0.91 | 2.74 | 0.012 | 0.45 | 0.095 |
| | | | | | | (1.25-6.00) | | (0.18-1.15) | |
| PWY0-1319 | CDP-diacylglycerol | 12403 | 11412 | 0.004 | 0.92 | 2.21 | 0.050 | 0.29 | 0.025 |
| | biosynthesis II | | | | | (1.00-4.89) | | (0.10-0.86) | |

Enzymes

| | | | | | | | | | |
|---|---|---|---|---|---|---|---|---|---|
| 1.1.1.18 | Inositol 2-dehydrogenase | 96 | 72 | 0.044 | 0.75 | 2.34 | 0.034 | 0.58 | 0.225 |
| | | | | | | (1.07-5.15) | | (0.24-1.40) | |
| 1.1.1.38, | Malate dehydrogenase | 1050 | 833 | 0.018 | 0.79 | 2.21 | 0.050 | 0.38 | 0.057 |
| 4.1.1.3 | (oxaloacetate- | | | | | (1.00-4.89) | | (0.14-1.03) | |
| | decarboxylating)l | | | | | | | | |
| | Oxaloacetate | | | | | | | | |
| | decarboxylase | | | | | | | | |
| 1.1.3.15 | (S)-2-hydroxy-acid | 104 | 77 | 0.045 | 0.74 | 2.00 | 0.088 | 0.38 | 0.057 |
| | oxidase | | | | | (0.90-4.44) | | (0.14-1.03) | |
| 1.14.13.39 | Nitric-oxide synthase | 123 | 94 | 0.012 | 0.77 | 1.97 | 0.090 | 0.40 | 0.067 |
| | (NADPH) | | | | | (0.90-4.34) | | (0.15-1.07) | |
| 1.17.7.3 | (E)-4-hydroxy- | 1337 | 1194 | 0.001 | 0.89 | 3.06 | 0.005 | 0.33 | 0.078 |
| | 3 -methylbut-2- | | | | | (1.40-6.70) | | (0.10-1.13) | |
| | enyl-diphosphate | | | | | | | | |
| | synthase (flavodoxin) | | | | | | | | |
| 1.2.7.1 | Pyruvate synthase | 94 | 76 | 0.036 | 0.80 | 1.94 | 0.097 | 0.59 | 0.244 |
| | | | | | | (0.89-4.22) | | (0.24-1.43) | |
| 1.2.7.3 | 2-oxoglutarate synthase | 94 | 52 | 0.044 | 0.55 | 2.72 | 0.012 | 0.29 | 0.051 |
| | | | | | | (1.24-5.94) | | (0.09-1.01) | |
| 1.2.7.8 | Indolepyruvate ferredoxin | 512 | 405 | 0.015 | 0.79 | 1.66 | 0.201 | 0.29(0.10-0.86) | 0.025 |
| | oxidoreductase | | | | | (0.76-3.59) | | | |
| 1.2.99.2 | Carbon-monoxide | 356 | 294 | 0.032 | 0.83 | 2.34 | 0.031 | 0.62 | 0.352 |
| | dehydrogenase (acceptor) | | | | | (1.08-5.06) | | (0.23-1.70) | |
| 2.5.1.47 | Cysteine synthase | 1287 | 1152 | 0.004 | 0.90 | 2.70 | 0.013 | 0.29 | 0.024 |
| | | | | | | (1.23-5.90) | | (0.10-0.85) | |
| 2.7.1.11 | 6-phosphofructokinase | 2123 | 1887 | 0.001 | 0.89 | 3.56 | 0.002 | 0.50 | 0.142 |
| | | | | | | (1.61-7.88) | | (0.20-1.26) | |
| 2.7.2.8 | Acetylglutamate kinase | 1324 | 1239 | 0.002 | 0.94 | 3.07 | 0.005 | 0.40 | 0.069 |
| | | | | | | (1.40-6.70) | | (0.15-1.08) | |
| 2.7.7.41 | Phosphatidate | 1058 | 866 | 0.049 | 0.82 | 2.77 | 0.011 | 0.45 | 0.149 |
| | cytidylyltransferase | | | | | (1.26-6.08) | | (0.15-1.33) | |
| 3.2.1.21 | Beta-glucosidase | 1079 | 919 | 0.031 | 0.85 | 2.05 | 0.069 | 0.46 | 0.106 |
| | | | | | | (0.95-4.43) | | (0.18-1.18) | |
| 3.2.1.51 | Alpha-L-fucosidase | 122 | 84 | 0.021 | 0.69 | 2.27 | 0.038 | 0.38 | 0.053 |
| | | | | | | (1.05-4.93) | | (0.14-1.01) | |
| 3.2.1.55 | Non-reducing end alpha- | 212 | 163 | 0.017 | 0.77 | 2.01 | 0.076 | 0.39 | 0.060 |
| | L-arabinofuranosidase | | | | | (0.93-4.33) | | (0.14-1.04) | |
| 3.4.21.89 | Signal peptidase I | 2488 | 2297 | 0.015 | 0.92 | 2.26 | 0.043 | 0.20 | 0.010 |
| | | | | | | (1.03-4.99) | | (0.06-0.68) | |
| 3.5.1.11 | Penicillin amidase | 162 | 55 | 0.016 | 0.34 | 2.80 | 0.010 | 0.43 | 0.132 |
| | | | | | | (1.27-6.16) | | (0.15-1.29) | |
| 3.5.1.24 | Choloylglycine hydrolase | 284 | 243 | 0.021 | 0.86 | 2.04 | 0.081 | 0.37 | 0.052 |
| | | | | | | (0.92-4.53) | | (0.14-1.01) | |
| 3.5.4.12 | dCMP deaminase | 307 | 251 | 0.041 | 0.82 | 2.60 | 0.016 | 0.46 | 0.166 |
| | | | | | | (1.19-5.66) | | (0.16-1.38) | |
| 4.2.1.126 | N-acetylmuramic acid | 255 | 199 | 0.041 | 0.78 | 3.03 | 0.006 | 0.46 | 0.103 |
| | 6- phosphate etherase | | | | | (1.38-6.68) | | (0.18-1.17) | |
| 4.6.1.12 | 2-C-methyl-D-erythritol | 1165 | 978 | 0.022 | 0.84 | 2.04 | 0.071 | 0.21 | 0.012 |
| | 2,4-cyclodiphosphate | | | | | (0.94-4.41) | | (0.06-0.71) | |
| | synthase | | | | | | | | |
| 4.99.1.3 | Sirohydrochlorin | 91 | 74 | 0.030 | 0.81 | 2.00 | 0.096 | 0.39 | 0.063 |
| | cobaltochelatase | | | | | (0.88-4.50) | | (0.14-1.05) | |
| 5.3.1.14 | L-rhamnose isomerase | 424 | 267 | 0.009 | 0.63 | 2.80 | 0.010 | 0.38 | 0.054 |
| | | | | | | (1.28-6.09) | | (0.14-1.02) | |
| 6.3.5.1 | NAD(+) synthase | 198 | 163 | 0.033 | 0.82 | 1.99 | 0.082 | 0.58 | 0.233 |
| | (glutaminehydrolyzing) | | | | | (0.92-4.30) | | (0.24-1.41) | |
| 6.3.5.11 | Cobyrinate a,c-diamide | 260 | 189 | 0.021 | 0.73 | 3.61 | 0.001 | 0.49 | 0.134 |
| | synthase (glutamine- | | | | | (1.65-7.88) | | (0.19-1.25) | |
| | hydrolyzing) | | | | | | | | |

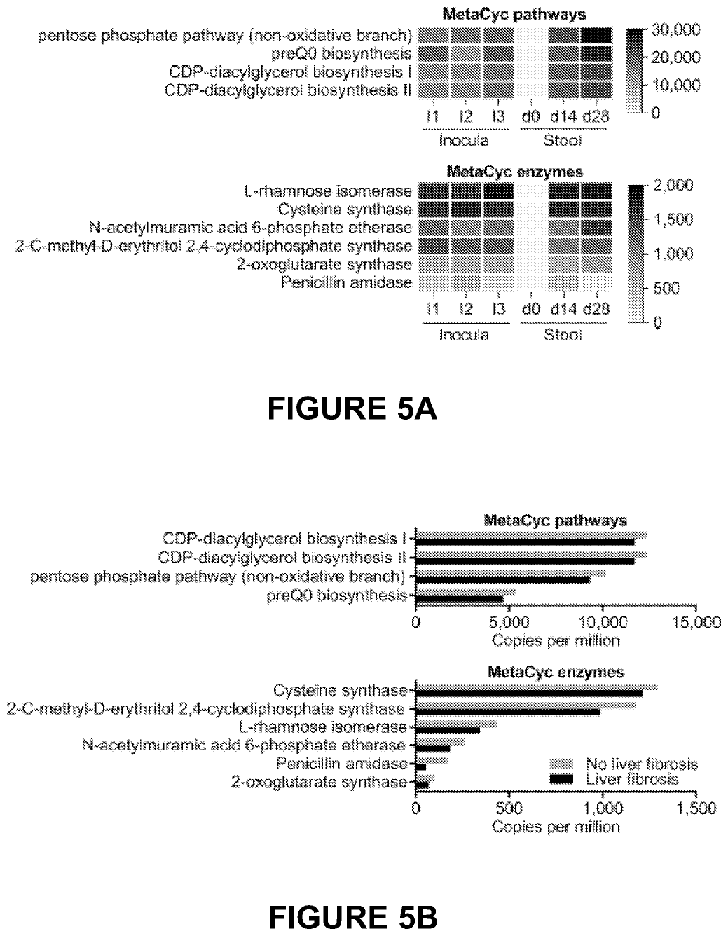

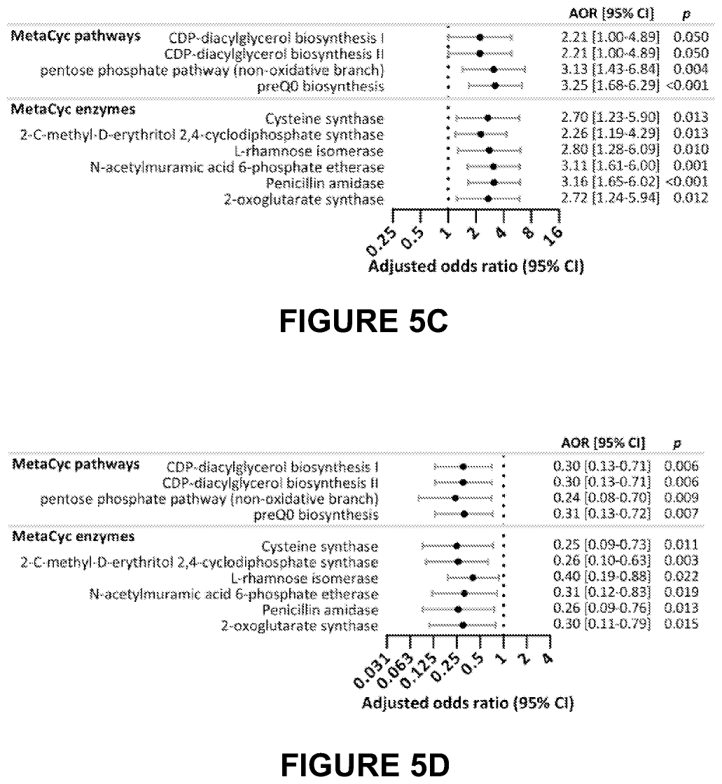

The stool metagenomic functions enriched in GF-MCD-B mice after bacterial inoculation were then characterized. Ninety-four MetaCyc pathways and 328 MetaCyc enzymes were detected in all three bacterial inocula and also enriched in all ten GF-MCD-B mice at days 14 and 28 after first inoculation. Integrating both human and mouse datasets, four pathways and six enzymes were enriched in the stool of mice after bacterial inoculation (FIG. 5A), significantly depleted in the cohort subjects with liver fibrosis (FIG. 5B), significantly associated with liver fibrosis when detected at low abundance in the cohort subjects (FIG. 5C), and significantly associated with protection against liver fibrosis when detected at high abundance in the cohort subjects (FIG. 5D). The four MetaCyc pathways included pentose phosphate pathway (non-oxidative branch) I (AOR(low)=3.13 [195% CI=1.43-6.84], p=0.004; AOR(high)=0.24 [195% CI=0.08-0.70], p=0.009), preQ0 biosynthesis (AOR(low)=3.25 [95% CI=1.68-6.29], p=<0.001; AOR(high)=0.31 [195% CI=0.13-0.72], p=0.007) and cytidine diphosphate (CDP)-diacylglycerol biosynthesis pathways I and II (AOR(low)=2.21 [95% CI=1.00-4.89], p=0.050; AOR(high)=0.30 [95% CI=0.13-0.71], p=0.006). The six enzymes included penicillin amidase (AOR(low)=3.16 [95% CI=1.65-6.02], p=<0.001; AOR(high)=0.26 [95% CI=0.09-0.76], p=0.013), cysteine synthase (AOR(low)=2.70 [95% CI=1.23-5.90], p=0.013; AOR(high)=0.25 [95% CI=0.09-0.73], p=0.011), 2-C-methyl-D-erythritol 2,4-cyclodiphosphate synthase (AOR(low)=2.26 [95% CI=1.19-4.29], p=0.013; AOR(high)=0.26 [95% CI=0.10-0.63], p=0.003), L-rhamnose isomerase (AOR(low)=2.80 [95% CI=1.28-6.09], p=0.010; AOR(high)=0.40 [95% CI=0.19-0.88],p=0.022), N-acetylmuramic acid 6-phosphate esterase (AOR(low)=3.11 [95% CI=1.61-6.00], p=0.001; AOR(high)=0.31 [95% CI=0.12-0.83], p=0.019) and 2-oxoglutarate synthase (AOR(low)=2.72 [95% CI=1.24-5.94], p=0.012; AOR(high)=0.30 [95% CI=0.11-0.79], p=0.015) (FIGS. 5C and 5D).

Cross-talk between the gut microbiome, host/bacterial metabolism, and immunity have been implicated in NAFLD pathology. Some studies have demonstrated the ability of specific bacterial metabolites to modulate hepatic metabolism, immune responses and transcriptional profiles. Metagenomic sequencing data from the inocula and stool of mice before and after bacterial inoculation identified pathways and enzymes associated with the presence of the bacterial consortium. To further determine the relevance of these pathways and enzymes in protecting against liver fibrosis, pathways and enzymes negatively associated with liver fibrosis in the human cohort were also identified. Comparative analysis of these two datasets, identified potential mechanisms of liver fibrosis prevention driven by bacterial treatment and of relevance to human disease. Following administration of the bacterial consortium, there was enrichment of the pentose phosphate pathway (non-oxidative branch) I, preQ0 biosynthesis and CDP-diacylglycerol biosynthesis pathways I and II. The non-oxidative branch is the reversible second step of the pentose phosphate pathway, a major metabolic pathway. Many bacteria lack the crucial transaldolase enzyme required for pathway I, and therefore use an alternative pathway II that involves alternate enzymes and intermediates. The implications of differing bacterial pentose phosphate non-oxidative pathways on the host remains to be determined. The preQ0 biosynthesis pathway is absent in mammals. The downstream products of preQ0 are used by both bacteria and mammals for post-translational Q-modification of tRNAs, which maintains optimal efficiency of protein translation speed and accuracy, and protects against oxidative stress in the mouse liver.⁴² In bacteria, CDP-diaglycerol is a lipid precursor to various phospholipids, including phosphatidylserine, phosphatidylglycerol, and cardiolipin. Therefore, enrichment of this pathway reflects crucial bacterial metabolic processes.

In addition to whole pathways, there were also 6 enzymes of interest enriched upon administration of the bacterial consortium, several of which are involved in broadly occuring microbial metabolic processes. L-rhamnose isomerase is involved in the microbial metabolism of L-rhamnose sugars, found in the pectin of plants and in bacterial cell walls. This enzyme catalyzes the first step of the pathway, which is a reversible conversion of L-rhamnose to L-rhamnulose.⁴⁴ N-acetylmuramic acid 6-phosphate etherase is involved in the catabolic recycling of peptidoglycan, an essential cell wall component for almost all bacteria. 2-C-methyl-D-erythritol 2,4-cyclodiphosphate synthase is involved in the microbial synthesis of isopentenyl pyrophosphate (IPP) and dimethylallyl pyrophosphate (DMAPP) through the methylerythritol phosphate pathway I, an essential metabolic pathway of bacteria. The synthesis of IPP and DMAPP occurs through a different pathway in humans, with differing intermediate metabolites. 2-oxoglutarate synthase is involved in multiple variations of the TCA cycle of respiration, including reverse TCA cycles. Penicillin amidase catalyzes the hydrolysis of various β-lactam antibiotics, and is structurally and phylogenetically related to bile salt hydrolase enzymes. Lastly, cysteine synthase is part of a de novo pathway of cysteine synthesis involving a two-step conversion of L-serine to L-cysteine. The integrated functional metagenome analysis identified cysteine synthase as a potential mediator of liver fibrosis protection in humans. This enzyme is part of a de novo pathway of cysteine synthesis, that is absent in mammals and involves a two-step conversion of L-serine to L-cysteine.

### Example 5

### Contribution of Amino Acids to the Anti-Fibrotic Effect of the Bacterial Consortium

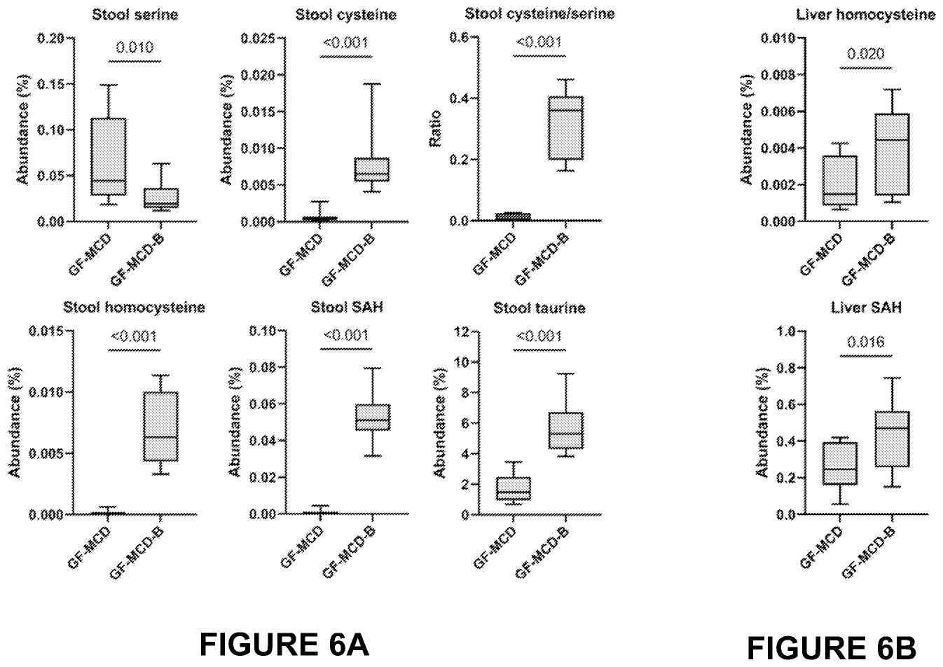

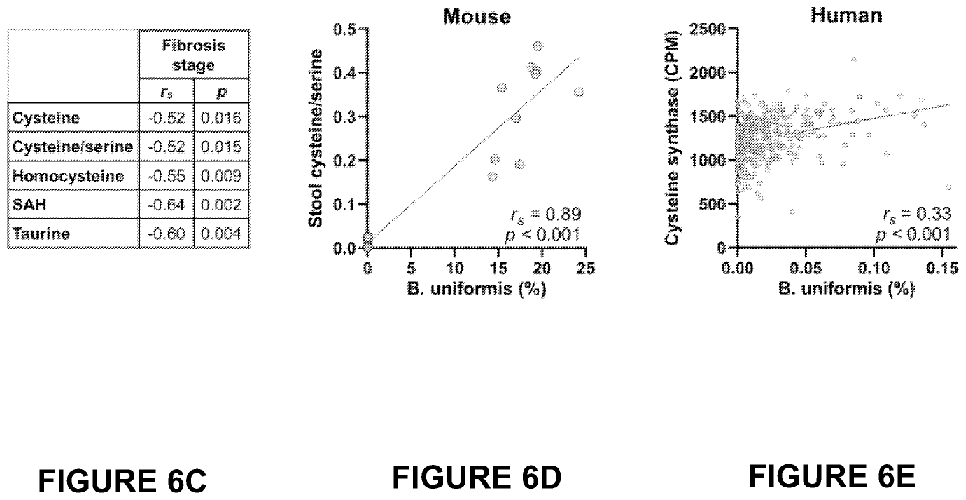

To determine whether the bacterial inoculations altered host levels of cysteine, serine and other related amino acids. comprehensive profiling of 37 amino acids was performed on stool, cecum and liver tissue collected from GF-MCD and GF-MCD-B mice at necropsy. The largest differences in abundance in GF-MCD-B mice, among all 37 amino acids measured, were increases in relative cysteine levels (FC=14.93, p<0.001) and in cysteine-to-serine ratios (FC=25.92, p<0.001), indicating a strong activation of cysteine synthase following bacterial inoculation (Table 4; FIG. 6A). Large increases were also observed in stool levels of the downstream amino acids of this pathway such as homocysteine (FC=93.80, p<0.001), S-adenosylhomocysteine (SAH) (FC=75.73, p<0.001) and taurine (FC=3.63, p<0.001) (FIG. 6A). The same strong increases in levels of cysteine (FC=11.58, p<0.001), cysteine-to-serine ratio (FC=38.85, p<0.001), homocysteine (FC=76.57, p<0.001), SAH (FC=26.85, p<0.001) and taurine (FC=2.65, p<0.001) were also observed in the cecum. TABLE 4 summarizes changes in the relative abundance of amino acids in stool, cecal content and liver in GF-MCD-B mice compared to GF-MCD mice. Significant differences in the relative abundance of each amino acid between GF-MCD and GF-MCD-B was assessed by Mann-Whitney test. For those amino acids showing significant changes in stool, abundance changes were also assessed in cecal content and liver. In TABLE 4, ‘FC’ denotes fold change (GF-MCD-B relative to GF-MCD) and ‘NC’ not changed significantly.

TABLE 4

| | Stool | | Cecal Content | | Liver | |
|---|---|---|---|---|---|---|
| Amino Acid | FC | p | FC | p | FC | p |
| Homocysteine | 93.80 | <0.001 | 76.57 | <0.001 | 2.96 | 0.020 |
| S-adenosyl- | 75.73 | <0.001 | 26.85 | <0.001 | 1.91 | 0.016 |
| homocysteine | | | | | | |
| Cysteine/serine | 25.92 | <0.001 | 38.85 | <0.001 | NC | |
| ratio | | | | | | |
| Cysteine | 14.93 | <0.001 | 11.58 | <0.001 | NC | |
| Taurine | 3.63 | <0.001 | 2.65 | <0.001 | NC | |
| Acetylcholine | 2.89 | 0.001 | 2.88 | <0.001 | NC | |
| Creatinine | 1.37 | 0.020 | NC | | NC | |
| Serine | −2.32 | 0.010 | −3.54 | <0.001 | NC | |
| Arginine | −2.33 | 0.029 | −1.22 | 0.010 | NC | |
| Phenylalaine | −2.41 | 0.006 | −3.20 | <0.001 | 1.48 | 0.029 |
| Tyrosine | −2.41 | 0.010 | −2.47 | <0.001 | NC | |
| Methionine | −2.46 | 0.002 | −3.49 | <0.001 | NC | |
| Sulfoxide | | | | | | |
| Cystine | −2.47 | 0.036 | −2.24 | 0.006 | NC | |
| Glutamine | −2.77 | 0.001 | −2.69 | <0.001 | 1.64 | 0.004 |
| Aspartic acid | −3.19 | 0.002 | −3.06 | <0.001 | NC | |
| Isoleucine | −3.49 | <0.001 | −3.91 | <0.001 | NC | |
| Leucine | −4.02 | <0.001 | −3.38 | 0.003 | NC | |
| Threonine | −8.06 | <0.001 | −9.72 | <0.001 | NC | |
| Asparagine | −12.59 | <0.001 | −44.55 | <0.001 | 1.22 | 0.043 |
| Glutamic acid | NC | | 1.85 | 0.006 | NC | |
| Choline | NC | | 1.26 | 0.002 | NC | |
| Histidine | NC | | −1.73 | 0.001 | NC | |
| Lysine | NC | | −2.16 | <0.001 | NC | |
| Valine | NC | | −3.05 | <0.001 | NC | |
| Glycine | NC | | NC | | 1.79 | 0.010 |
| Tryptophan | NC | | −2.54 | <0.001 | 1.49 | 0.010 |
| Kynurenine | NC | | NC | | −1.66 | 0.036 |
| Kynurenine/ | NC | | 2.65 | 0.001 | −2.73 | 0.001 |
| tryptophan ratio | | | | | | |
| Carnitine | NC | | NC | | −1.23 | 0.024 |
| Hypotaurine | NC | | NC | | −1.53 | 0.010 |
| 4-hydroxyproline | NC | | NC | | NC | |
| Alanine | NC | | NC | | NC | |
| Citrulline | NC | | NC | | NC | |
| Creatine | NC | | NC | | NC | |
| Methionine | NC | | NC | | NC | |
| Methionine | NC | | NC | | NC | |
| Sulfone | | | | | | |
| Ornithine | NC | | NC | | NC | |
| Proline | NC | | NC | | NC | |
| Sarcosine | NC | | NC | | NC | |

Finally, increased levels of homocysteine (FC=2.96, p=0.020) and SAH (FC=1.91, p=0.016) were also found in the livers of GF-MCD-B mice (FIG. 6B). Importantly strong negative correlations were observed between fibrosis scores and the cysteine-to-serine ratio (rₛ=−0.52, p=0.015), abundance of cysteine (rₛ=−0.52, p=0.016), homocysteine (rₛ=−0.55, p=0.009), SAH (rₛ=−0.64, p=0.002) and taurine (rₛ=−0.60, p=0.004) (FIG. 6C). In the cecum, similar significant negative correlations with fibrosis scores were observed (cysteine-to-serine ratio: rₛ=−0.48, p=0.028; cysteine: rₛ=−0.46, p=0.036; homocysteine: rₛ=−0.53; p=0.014; SAH: rₛ=−0.58, p=0.006; taurine: rₛ=−0.61, p=0.003). To identify which bacterial species in the consortium contributed to increased cysteine synthesis, we interrogated the whole genome annotations of the six colonizing species. While all six of the colonizing strains possessed the cysteine synthase enzyme, only Bacteroides uniformis ATCC 8492 also possessed serine acetyltransferase, the first enzyme of the two-step de novo cysteine synthesis pathway (TABLE 5). To determine the relevance of the selected strains in the consortium to our human cohort, we also retrieved the genome sequences for all other strains detected in the human cohort and belonging to the six colonizing species. This confirmed that the presence of cysteine synthase was universal across all strains interrogated. On the contrary, serine acetyltransferase was absent in almost all strains, except for Bacteroides uniformis ATCC 8492 as described, a second, rare Bacteroides uniformis strain (dnLKV2), and 3 rare Bacteroides ovatus strains (3_8_47FAA, str. 3725 D1 iv, str. 3725 D9 iii). In conclusion, we find that Bacteroides uniformis ATCC 8492, the only strain in our consortium able to perform the full de novo cysteine synthesis pathway, is also likely a major contributor to the pathway in the human cohort, as all other strains with an appreciable detection rate lack the serine acetyltransferase enzyme. The role of Bacteroides uniformis in the observed activation of the cysteine synthesis pathway was further suggested by a strong correlation between Bacteroides uniformis abundance and cysteine-to-serine ratios in stool (FIG. 6D, rₛ=0.89, p<0.001), while none of the other bacterial species showed a significant correlation. In stool from the 340 cohort subjects, Bacteroides uniformis abundance also correlated with abundance of cysteine synthase (FIG. 6E), further suggesting a contribution of Bacteroides uniformis to cysteine synthase activity in the gut.

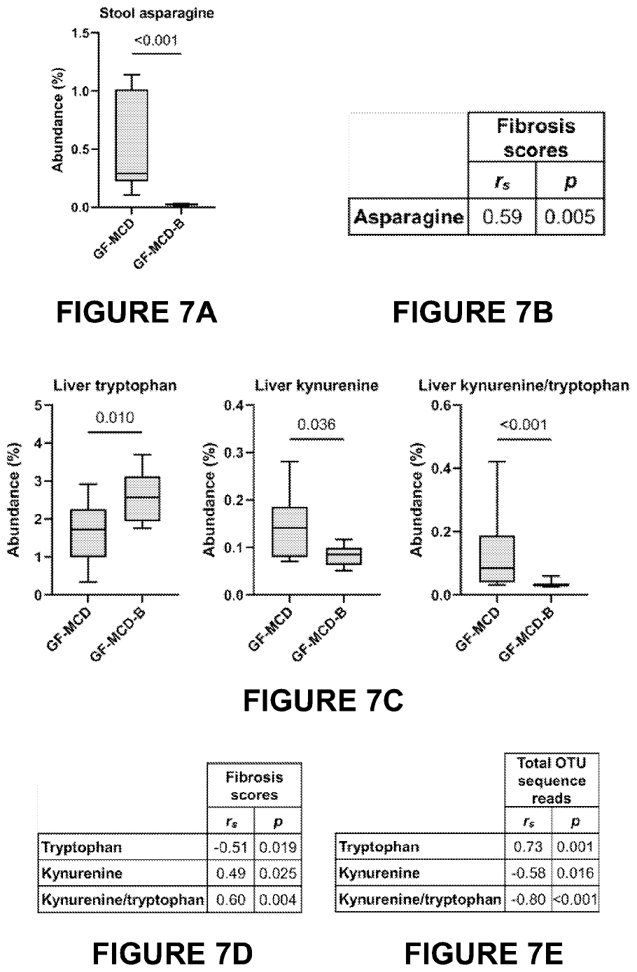

The other major change in amino acid abundance was a sharp decrease in asparagine levels in the stool (FC=−12.59, p<0.001, FIG. 7A) and cecum (FC=−44.55, p<0.001) of GF-MCD-B mice. Liver fibrosis scores significantly correlated with asparagine levels in both the stool (rₛ=0.59, p=0.005) and cecum (rₛ=0.61, p=0.003) (FIG. 7B). Based on whole genome annotation data, all six colonizing species possessed at least one copy of the asparaginase enzyme responsible for L-asparagine degradation. The presence of the asparaginase gene was universal across all strains detected in the human cohort and belonging to the six colonizing species.

TABLE 5 summarizes gene copy numbers of amino acid-metabolizing enzymes in all bacterial strains detected in the human cohort and belonging to the six species successfully colonizing the mouse gut after inoculation. For each strain, raw genome sequencing files were retrieved from the ATCC Genome Portal if available, or the NCBI data hub, and annotated with the prokka pipeline, available on the Proksee online tool, to determine the presence of each enzyme in each bacterial strain genome. Genomes from the ATCC Genome Portal lack accession numbers, as one single genome is provided for each strain. Genome sequences were not available for unclassified strain-level data, nor for Bacteroides ovatus CL02T12CO4. Strains are sorted by detection rate in the 340 human study participants. Strains in bold were used for the bacterial consortium in the in vivo study. For each species, the most abundant strain was selected if commercially available.

TABLE 5

| | | | | | Serine | | |
|---|---|---|---|---|---|---|---|
| | | | | | acetyl- | Cysteine | Aspara- |
| | Detec- | Mean | | | transferase | synthase | ginase |
| | tion | Abun- | | Genome | (EC | (EC | (EC |
| | Rate | dance | Source | Accession Number | 2.3.1.30) | 2.5.1.47) | 3.5.1.1) |
| Bacteroides | 76% | 0.19% | | — | — | — | — |
| caccae | | | | | | | |
| Bacteroides | 51% | 0.16% | ATCC | N/A | 0 | 2 | 3 |
| caccae | | | | | | | |
| ATCC 43185 | | | | | | | |
| Bacteroides | 19% | 0.03% | NCBI | GCF_018292205.1 | 0 | 2 | 3 |
| caccae | | | | | | | |
| CL03T12C61 | | | | | | | |
| Bacteroides | 6% | 0.00% | — | — | — | — | — |
| caccae | | | | | | | |
| (unclassified) | | | | | | | |
| Bacteroides | 61% | 0.22% | | — | — | — | — |
| finegoldii | | | | | | | |
| Bacteroides | 55% | 0.21% | NCBI | GCF_000156195.1 | 0 | 2 | 3 |
| finegoldii | | | | | | | |
| DSM 17565 | | | | | | | |
| Bacteroides | 6% | 0.00% | NCBI | GCF_000304195.1 | 0 | 3 | 3 |
| finegoldii | | | | | | | |
| CL09T03C10 | | | | | | | |
| Bacteroides | 94% | 0.63% | | — | — | — | — |
| ovatus | | | | | | | |
| Bacteroides | 46% | 0.28% | NCBI | GCF_000178275.1 | 0 | 2 | 3 |
| ovatus SD | | | | | | | |
| CMC 3f | | | | | | | |
| Bacteroides | 33% | 0.23% | — | — | — | — | — |
| ovatus | | | | | | | |
| CL02T12C04 | | | | | | | |
| Bacteroides | 6% | 0.03% | ATCC | N/A | 0 | 2 | 3 |
| ovatus | | | | | | | |
| ATCC 8483 | | | | | | | |
| Bacteroides | 4% | 0.04% | NCBI | GCF_000218325.1 | 1 | 2 | 3 |
| ovatus | | | | | | | |
| 3_8_47FAA | | | | | | | |
| Bacteroides | 2% | 0.03% | NCBI | GCF_018492845.1 | 0 | 3 | 3 |
| ovatus | | | | | | | |
| CL03T12C18 | | | | | | | |
| Bacteroides | 2% | 0.01% | NCBI | GCF_000699725.1 | 1 | 3 | 3 |
| ovatus str. | | | | | | | |
| 3725 D1 iv | | | | | | | |
| Bacteroides | 1% | 0.00% | NCBI | GCF_000699665.1 | 1 | 2 | 3 |
| ovatus str. | | | | | | | |
| 3725 D9 iii | | | | | | | |
| Bacteroides | 92% | 2.24% | | — | — | — | — |
| uniformis | | | | | | | |
| Bacteroides | 40% | 0.88% | NCBI | GCF_900107315.1 | 1 | 2 | 2 |
| uniformis | | | | | | | |
| ATCC 8492 | | | | | | | |
| Bacteroides | 19% | 0.39% | — | — | — | — | — |
| uniformis | | | | | | | |
| (unclassified) | | | | | | | |
| Bacteroides | 14% | 0.43% | NCBI | GCF_000699825.1 | 0 | 2 | 2 |
| uniformis str. | | | | | | | |
| 3978 T3 ii | | | | | | | |
| Bacteroides | 8% | 0.28% | NCBI | GCF_000699885.1 | 0 | 3 | 2 |
| uniformis str. | | | | | | | |
| 3978 T3 i | | | | | | | |
| Bacteroides | 6% | 0.11% | NCBI | GCF_018292165.1 | 0 | 2 | 2 |
| uniformis | | | | | | | |
| CL03T12C37 | | | | | | | |
| Bacteroides | 6% | 0.15% | NCBI | GCF_000403175.1 | 1 | 2 | 2 |
| uniformis | | | | | | | |
| dnLKV2 | | | | | | | |
| Parabacteroides | 84% | 0.49% | | — | — | — | — |
| distasonis | | | | | | | |
| Parabacteroides | 27% | 0.13% | — | — | — | — | — |
| distasonis | | | | | | | |
| (unclassified) | | | | | | | |
| Parabacteroides | 26% | 0.17% | NCBI | GCF_000307435.1 | 0 | 1 | 1 |
| distasonis | | | | | | | |
| CL09T03C24 | | | | | | | |
| Parabacteroides | 13% | 0.06% | ATCC | N/A | 0 | 1 | 1 |
| distasonis | | | | | | | |
| ATCC 8503 | | | | | | | |
| Parabacteroides | 8% | 0.08% | NCBI | GCF_018292145.1 | 0 | 1 | 1 |
| distasonis | | | | | | | |
| CL03T12C09 | | | | | | | |
| Parabacteroides | 5% | 0.03% | NCBI | GCF_000699765.1 | 0 | 1 | 1 |
| distasonis str. | | | | | | | |
| 3999B T(B) 6 | | | | | | | |
| Parabacteroides | 2% | 0.01% | NCBI | GCF_000699905.1 | 0 | 1 | 1 |
| distasonis str. | | | | | | | |
| 3999B T(B) 4 | | | | | | | |
| Parabacteroides | 2% | 0.01% | NCBI | GCF_000699745.1 | 0 | 1 | 1 |
| distasonis str. | | | | | | | |
| 3776 Po2 i | | | | | | | |
| Parabacteroides | 0% | 0.00% | NCBI | GCF_000699785.1 | 0 | 1 | 1 |
| distasonis str. | | | | | | | |
| 3776 D15 i | | | | | | | |
| Parabacteroides | 0% | 0.00% | NCBI | GCF_000699805.1 | 0 | 1 | 1 |
| distasonis str. | | | | | | | |
| 3776 D15 iv | | | | | | | |
| Alistipes | 66% | 0.36% | | — | — | — | — |
| onderdonkii | | | | | | | |
| Alistipes | 35% | 0.26% | NCBI | GCF_006542645.1 | 0 | 1 | 1 |
| onderdonkii | | | | | | | |
| subsp. vulgaris | | | | | | | |
| Alistipes | 31% | 0.09% | NCBI | GCF_000374505.1 | 0 | 1 | 1 |
| onderdonkii | | | | | | | |
| DSM 19147 | | | | | | | |

Finally, a concomitant increase in tryptophan (FC=1.49, p=0.010) and decrease in its product kynurenine (FC=−1.66, p=0.036), was observed in the livers of GF-MCD-B mice, but not in the stool nor cecum, suggesting decreased conversion of tryptophan to kynurenine specifically in the liver (FIG. 7C). This was confirmed by a strong decrease in the kynurenine-to-tryptophan ratio (FC=−2.73, p=0.001). Most importantly, hepatic levels of tryptophan negatively correlated with liver fibrosis scores (rₛ=−0.51, p=0.019), while kynurenine levels and kynurenine-to-tryptophan ratios positively correlated with liver fibrosis scores (rₛ=0.49, p=0.025 and rₛ=0.60, p=0.004, respectively) (FIG. 7D). Hepatic levels of tryptophan were associated with overall bacterial load in the liver, as the number of total OTU sequence reads in the liver positively correlated with tryptophan levels (rₛ=0.73, p=0.001), while kynurenine levels and kynurenine-to-tryptophan ratios negatively correlated with total OTU sequence reads (rₛ=−0.58, p=0.016 and rₛ=−0.80, p<0.001, respectively) (FIG. 7E).

### Discussion

Cysteine is a sulfur-containing, semi-essential amino acid and the de novo pathway is absent in mammalian cells. Instead, mammals generate cysteine by the reverse transsulfuration pathway, involving conversion of homocysteine to cysteine. Amino acids are key modulators of metabolism, and emerging as a potential therapeutic strategy to target multifactorial, metabolic diseases such as NAFLD. Comprehensive profiling of 37 amino acids in the stool, cecum and liver of MCD-fed GF mice with or without bacteria treatment was performed using ultra-high-resolution mass spectrometry. Enrichment of cysteine synthase in the stool and cecum upon bacterial inoculation was indeed accompanied by increased conversion of serine to cysteine, indicating activation of the de novo synthesis pathway in the gut of the treated mice. Increased stool and cecal levels of homocysteine, SAH, and taurine were also observed. Most importantly, stool levels of cysteine, cysteine/serine ratio, homocysteine, SAH and taurine all negatively correlated with fibrosis scores in the mice, confirming a protective role of these amino acids against liver fibrosis. Homocysteine and SAH levels were also increased in the liver. Taurine is a major downstream product of cysteine oxidation, which protects against features of NAFLD in vivo, including inflammation, steatosis, fibrosis and oxidative stress. In vivo studies have suggested a role for N-acetylcysteine, an acetylated precursor to cysteine, in preventing hepatic lipid accumulation, oxidative stress and inflammation in NAFLD.

Overall, treatment with the bacterial consortium may confer protection against liver fibrosis due to activation of cysteine synthase and subsequent altered metabolism of cysteine-related amino acids. Cysteine also gives rise to two other important downstream products, glutathione and hydrogen sulfide, with protective effects against oxidative stress, inflammation, NAFLD and fibrosis. As the bacterial consortium was also detected in the liver, bacterial-dependent cysteine metabolism may contribute to liver fibrosis protection directly in the intrahepatic environment. Amongst all bacteria in the consortium, increased activity of this pathway was likely dependent upon the presence of Bacteroides uniformis, which was the only strain to possess both genes in the de novo synthesis pathway. Confirming this hypothesis, a strong correlation was observed between Bacteroides uniformis abundance and cysteine-to-serine ratios in mouse stool, as well as correlation between Bacteroides uniformis and the cysteine synthase enzyme in human stool. This is the first study demonstrating a protective role against liver fibrosis and implicating the bacteria cysteine synthase function.

Another major amino acid change observed following bacterial treatment, was a strong depletion of asparagine in stool and cecum, which correlated with reduced liver fibrosis severity. Asparagine is sourced from the diet or synthesized by asparaginase synthetase, and metabolised by asparaginase. As possession of these genes varies between microbes, the gut microbiome composition is thought to influence the enzyme activity and asparagine levels. All six colonizing species were predicted to possess asparaginase activity, which hydrolyzes asparagine to aspartic acid and ammonia. Gut microbial communities with high Bacteroides abundance are associated with increased asparaginase activity.

Decreased conversion of tryptophan to kynurenine specifically in the liver correlated with reduced liver fibrosis severity. Interestingly, tryptophan levels positively correlated with hepatic bacterial load, suggesting that bacteria presence in the liver mediated the loss of conversion to kynurenine. Tryptophan is an essential amino acid obtained from the diet. The majority of tryptophan in mammalian cells is catabolized through the kynurenine pathway. The rate-limiting first step is initiated by tryptophan 2,3-dioxygenase (TDO) in the liver, and is activated by tryptophan and corticosteroids. Certain bacteria, including Bacteroides species, can also catabolize tryptophan through other pathways, predominantly the indole pathway. Therefore, intrahepatic bacteria may attenuate the kynurenine pathway by indirectly suppressing TDO activity, or by redirecting tryptophan catabolism through bacterial pathways instead of the kynurenine pathway. Elevated stool levels of kynurenine were observed in patients with NAFLD, and fecal microbiota transplant from NAFLD patients to GF mice resulted in increased serum kynurenine levels. Interestingly, microbial-derived indole and its derivatives can also modulate liver metabolism and immune responses.

In conclusion, taxonomic changes were associated with reduced risk of liver fibrosis in a high-risk population. Based on these findings, the abilities of 8 bacteria to prevent NAFLD-related liver fibrosis were tested in vivo, strongly suggesting that subsets of these bacteria are hepatoprotective, as well. Treatment with the bacterial consortium conferred protection against liver fibrosis in a diet-induced, GF mouse model of steatohepatitis. Presence of the bacterial species was detected in both the stool and liver. Comparative analysis of metagenomic functional profiles in human and mice identified pathways and enzymes, likely to mediate the protective effect of the bacterial consortium against liver fibrosis, of relevance to the human disease. A role for cysteine synthase activation was further confirmed by amino acid profiling. Other liver fibrosis-associated amino acids pathways included asparaginase activity in stool and conversion of tryptophan to kynurenine in liver. These findings support further development of live biotherapeutic products for the prevention of liver fibrosis in NAFLD, and provide novel insights into their mechanism of action.

Although the invention has been described with reference to the above examples, it will be understood that modifications and variations are encompassed within the spirit and scope of the invention. Accordingly, the invention is limited only by the following claims.

1. A composition comprising two or more bacteria selected from the group consisting of:
(a) Bacteroides finegoldii;
(b) Bacteroides caccae;
(c) Bacteroides ovatus,
(d) Bacteroides uniformis,
(e) Parabacteroides distasonis,
(f) Alistipes onderdonkii,
(g) Anaerobutyricum hallii,
(h) Gemmiger formicilis, and
(i) a combination thereof.

2. The composition of claim 1, wherein the composition comprises at least three, at least four, at least five, at least six, at least seven or eight of the bacteria.

3-7. (canceled)

8. The composition of claim 1, wherein the composition comprises a functional mutant of the Bacteroides finegoldii, a functional mutant of the Bacteroides caccae, a functional mutant of the Bacteroides ovatus, a functional mutant of the Bacteroides uniformis, a functional mutant of the Parabacteroides distasonis, a functional mutant of the Alistipes onderdonkii, a functional mutant of the Anaerobutyricum hallii, a functional mutant of the Gemmiger formicilis, or a combination thereof.

9. The composition of claim 1, wherein:
(a) the Bacteroides finegoldii comprises at least 90% identity to the nucleotide sequence of strain DSM 17565 deposited with the Deutsche Sammlung von Mikroorganismen und Zellkulturen (DSMZ);
(b) the Bacteroides caccae comprises at least 90% identity to the nucleotide sequence of strain DSM 19024 deposited with the DSMZ;
(c) the Bacteroides ovatus comprises at least 90% identity to the nucleotide sequence of strain 1896 DSM deposited with the DSMZ;
(d) the Bacteroides uniformis comprises at least 90% identity to the nucleotide sequence of strain DSM 6597 deposited with the DSMZ;
(e) the Parabacteroides distasonis comprises at least 90% identity to the nucleotide sequence of strain DSM 20701 deposited with the DSMZ;
(f) the Alistipes onderdonkii comprises at least 90% identity to the nucleotide sequence of strain DSM 32839 deposited with the DSMZ;
(g) the Anaerobutyricum hallii comprises at least 90% identity to the nucleotide sequence of strain DSM 3353 deposited with the DSMZ;
(h) the Gemmiger formicilis comprises at least 90% identity to the nucleotide sequence of strain ATCC 27749 deposited with the American Type Culture Collection (ATCC); or
(i) a combination thereof.

10. The composition of claim 1, wherein the composition comprises between about 1×10⁷ and 1×10¹⁰ colony forming units (CFU) of live bacteria.

11. The composition of claim 1, wherein:
(a) a relative abundance of Bacteroides finegoldii among bacteria of the composition is between about 8% and 30%;
(b) a relative abundance of Bacteroides caccae among the bacteria of the composition is between about 11% and 45%;
(c) a relative abundance of Bacteroides ovatus among the bacteria of the composition is between about 7% and 27%;
(d) a relative abundance of Bacteroides uniformis among the bacteria of the composition is between about 3% and 10%;
(e) a relative abundance of Parabacteroides distasonis among the bacteria of the composition is between about 8% and 31%;
(f) a relative abundance of Alistipes onderdonkii among the bacteria of the composition is between about 5% and 21%;
(g) a relative abundance of Anaerobutyricum hallii among the bacteria of the composition is between about 1% and 6%;
(h) a relative abundance of Gemmiger formicilis among the bacteria of the composition is between about 4% and 16%; or
(i) a combination thereof.

12. The composition of claim 1, wherein at least about 50% of bacteria of the composition are Gram-negative.

13-14. (canceled)

15. A method of providing hepatoprotection or treating or preventing a disease or condition in a subject in need thereof comprising administering the composition of claim 1 to the subject,
wherein the disease or condition is selected from steatosis, nonalcoholic steatohepatitis (NASH), nonalcoholic fatty liver disease (NAFLD), liver fibrosis, cirrhosis of the liver, or a combination thereof, thereby providing hepatoprotection or treating the disease or condition.

16. The method of claim 15, wherein prior to the administration, the method further comprises determining the relative abundance of bacteria in the intestinal microbiome of the subject, and administering the composition to subject identified with bacterial depletion in the subject.

17. The method of claim 16, wherein identifying bacterial depletion comprises:
(i) measuring a relative abundance of Bacteroides finegoldii of less than about 0.2% in the intestinal microbiome in the subject;
(ii) measuring a relative abundance of Bacteroides caccae of less than about 0.1% in the intestinal microbiome in the subject;
(iii) measuring a relative abundance of Bacteroides ovatus of less than about 0.5% in the intestinal microbiome in the subject;
(iv) measuring a relative abundance of Bacteroides uniformis of less than about 2.0% in the intestinal microbiome in the subject;
(v) measuring a relative abundance of Parabacteroides distasonis of less than about 0.4% in the intestinal microbiome in the subject;
(vi) measuring a relative abundance of Alistipes onderdonkii of less than about 0.2% in the intestinal microbiome in the subject;
(vii) measuring a relative abundance of Anaerobutyricum hallii of less than about 1.0% in the intestinal microbiome in the subject;
(viii) measuring a relative abundance of Gemmiger formicilis of less than about 0.8% in the intestinal microbiome in the subject; or
(ix) a combination thereof.

18. The method of claim 16, wherein the method comprises at least two, at least three, at least four, at least five, at least six, at least seven, or each of (i)-(viii).

19. (canceled)

20. The method of claim 15, wherein the administering is repeated once every 3 to 10 days.

21. The method of claim 15, wherein the composition is administered orally.

22. The method of claim 15, further comprising administering a dietary supplement that supports the growth or maintenance of Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, or a combination thereof.

23. The method of claim 15, further comprising administering an antimicrobial agent which kills or slows the growth of a bacterium in the subject and which does not kill or slow the growth of bacteria of the composition.

24. The method of claim 15, wherein following the administration, the method further comprises determining the relative abundance of bacteria in the intestinal microbiome of the subject, thereby determining that the disease or condition has been treated in the subject.

25. The method of claim 24, wherein the bacteria and determining is selected from:
(i) determining that a relative abundance of Bacteroides finegoldii is greater than about 0.2% in the intestinal microbiome in the subject;
(ii) determining that a relative abundance of Bacteroides caccae is greater than about 0.1% in the intestinal microbiome in the subject;
(iii) determining that a relative abundance of Bacteroides ovatus is greater than about 0.5% in the intestinal microbiome in the subject;
(iv) determining that a relative abundance of Bacteroides uniformis is greater than about 2.0% in the intestinal microbiome in the subject;
(v) determining that a relative abundance of Parabacteroides distasonis is greater than about 0.4% in the intestinal microbiome in the subject;
(vi) determining that a relative abundance of Alistipes onderdonkii is greater than about 0.2% in the intestinal microbiome in the subject;
(vii) determining that a relative abundance of Anaerobutyricum hallii is greater than about 1.0% in the intestinal microbiome in the subject;
(viii) determining that a relative abundance of Gemmiger formicilis is greater than about 0.8% in the intestinal microbiome in the subject; or
(ix) a combination thereof.

26. The method of claim 15, further comprising following the administration:
a) measuring a level of serine and/or asparagine in stool of the subject, wherein:
i) measuring less than about 0.05% relative abundance of serine among amino acids and/or
ii) measuring less than 0.1% abundance among amino acids of asparagine, is indicative that the disease or condition has been treated in the subject,
b) measuring a level of cysteine, homocysteine, S-adenosylhomocysteine, tryptophan, taurine or a combination thereof in stool of the subject, wherein:
i) measuring greater than about 0.005% relative abundance of cysteine among amino acids,
ii) measuring a ratio of cysteine to serine of greater than about 0.2,
iii) measuring greater than 0.005% relative abundance among amino acids of homocysteine,
iv) measuring greater than 0.05% relative abundance among amino acids of S-adenosylhomocysteine,
vi) measuring greater than 3% relative abundance among amino acids of tryptophan, is indicative that the disease or condition has been treated in the subject,
and/or
c) measuring a level of tryptophan, kynurenine, homocysteine, S-adenosylhomocysteine or a combination thereof in liver tissue of the subject, wherein:
i) measuring greater than 3% relative abundance among amino acids of tryptophan in stool of the subject,
ii) measuring less than 0.15% relative abundance among amino acids of kynurenine in liver tissue of the subject,
iii) measuring a kynurenine to tryptophan ratio of less than 0.05 in liver tissue of the subject,
iv) measuring a greater than 0.005% relative abundance among amino acids of homocysteine in liver tissue of the subject, and/or
v) measuring a greater than 0.5% relative abundance among amino acids of S-adenosylhomocysteine in liver tissue of the subject, is indicative that the disease or condition has been treated in the subject.

27. The method of claim 15, further comprising, following the administration, measuring a parameter of a biosynthetic pathway selected from pentose phosphate pathway non-oxidative branch, diacylglycerol biosynthesis I, preQ0 biosynthesis, or CDP-diacylglycerol biosynthesis II in the stool of the subject, wherein:
i) measuring greater than 10000 copies per million of enzymes from the pentose phosphate pathway non-oxidative branch,
ii) measuring greater than 12000 copies per million of enzymes from the diacylglycerol biosynthesis I pathway,
iii) measuring greater than 5000 copies per million of enzymes from the preQ0 biosynthesis pathway, and/or
measuring greater than 12000 copies per million of enzymes from the CDP-diacylglycerol biosynthesis II pathway
is indicative that the disease or condition has been treated in the subject.

28. The method of claim 15, further comprising, following the administration, measuring a level of 2-oxoglutarate synthase, cysteine synthase, penicillin amidase, N-acetylmuramic acid 6-phosphate etherase, 2-C-methyl-D-erythritol 2,-4-cyclodiphosphate synthase, L-rhamnose isomerase, or a combination thereof in stool of the subject, wherein:
i) measuring greater than 90 copies per million of 2-oxoglutarate,
ii) greater than 1250 copies per million of cysteine synthase,
iii) measuring greater than 140 copies per million of penicillin amidase,
iv) measuring greater than 220 copies per million of N-acetylmuramic acid 6-phosphate etherase,
v) measuring greater than 1100 copies per million of 2-C-methyl-D-erythritol 2,4-cyclodiphosphate synthase, and/or
vi) measuring greater than 400 copies per million of L-rhamnose isomerase
is indicative that the disease or condition has been treated in the subject.

29. (canceled)

30. A method of identifying a subject as having a condition or being at risk of developing a disease or condition comprising:
i) detecting depletion of Bacteroides finegoldii, Bacteroides caccae, Bacteroides ovatus, Bacteroides uniformis, Parabacteroides distasonis, Alistipes onderdonkii, Anaerobutyricum hallii, Gemmiger formicilis, or a combination thereof in stool of the subject;
ii) detecting depletion of a biosynthetic pathway selected from pentose phosphate pathway non-oxidative branch, diacylglycerol biosynthesis I, preQ0 biosynthesis, or CDP-diacylglycerol biosynthesis II in stool of the subject;
iii) detecting depletion of 2-oxoglutarate synthase, cysteine synthase, penicillin amidase, N-acetylmuramic acid 6-phosphate etherase, 2-C-methyl-D-erythritol 2,-4-cyclodiphosphate synthase, L-rhamnose isomerase, or a combination thereof in stool of the subject;
iv) detecting depletion of cysteine, homocysteine, S-adenosylhomocysteine, tryptophan, or a combination thereof in stool of the subject,
v) detecting depletion of homocysteine, S-adenosylhomocysteine, kynurenine, or a combination thereof in liver tissue of the subject,
vi) detecting an increase in serine, asparagine, or a combination thereof in stool of the subject, or
vii) a combination thereof,
wherein the disease or condition is selected from steatosis, nonalcoholic steatohepatitis (NASH), nonalcoholic fatty liver disease (NAFLD), liver fibrosis, or cirrhosis of the liver, thereby identifying the subject as having the condition or being at risk of developing the condition.

31-35. (canceled)

36. The method of claim 30, further comprising administering to the subject identified as having the disease or condition a composition comprising two or more bacteria selected from the group consisting of: (a) Bacteroides finegoldii; (b) Bacteroides caccae; (c) Bacteroides ovatus, (d) Bacteroides uniformis, (e) Parabacteroides distasonis, (f) Alistipes onderdonkii, (g) Anaerobutyricum hallii, (h) Gemmiger formicilis, and (i) a combination thereof.
